(19) United States
(12) Patent Application Publication
(10) Pub. No.: US 2004/0170685 A1
(43) Pub. Date: Sep. 2, 2004

(54) Bioactive stents and methods for use thereof

(75) Inventors: Kenneth W. Carpenter, San Diego, CA (US); Huashi Zhang, San Diego, CA (US); Brendan J. McCarthy, Cardiff, CA (US); Istvan Szinai, San Diego, CA (US); William G. Turnell, San Diego, CA (US); Sindhu M. Gopalan, San Diego, CA (US)

Correspondence Address: GRAY CARY WARE & FREIDENRICH LLP, 4365 EXECUTIVE DRIVE, SUITE 1100, SAN DIEGO, CA, 92121-2133, US

(73) Assignee: MediVas, LLC, San Diego, CA (US)

(21) Appl. No.: 10/788,747

(22) Filed: Feb. 26, 2004

Related U.S. Application Data
(60) Provisional application No. 60/450,627, filed on Feb. 26, 2003.
(60) Provisional application No. 60/464,381, filed on Apr. 21, 2003.

(51) Int. Cl. A61K 31/7048; A61K 31/685

(52) U.S. Cl. 424/468; 514/78; 514/27; 514/509; 514/364; 514/565

(57) ABSTRACT

The present invention is based on the discovery that stents can be coated with biodegradable, bioactive polymers that promote endogenous healing processes at a site of stent implantation. The polymers biodegrade over time, releasing agents which establish or re-establish the natural healing process in an artery. Preferably, the stent is implanted at the time an artery is damaged, such at the time of angioplasty to protect the damaged artery against deleterious blood-borne factors that initiate proliferation of smooth muscle cells.

### RELATED APPLICATIONS

This application claims the benefit of priority under 35 U.S.C. § 119 of U.S. Serial No. 60/450,627, filed Feb. 26, 2003 and U.S. Serial No. 60/464,381, filed Apr. 21, 2003, the entire contents of each of which are incorporated herein by reference.

### FIELD OF THE INVENTION

The invention relates generally to implantable stents, and in particular to biodegradable polymer coated implantable stents that promote vascular healing.

### BACKGROUND INFORMATION

The normal endothelium, which lines blood vessels, is uniquely and completely compatible with blood. Endothelial cells initiate metabolic processes, like the secretion of prostacylin and endothelium-derived relaxing factor (EDRF), which actively discourage platelet deposition and thrombus formation in vessel walls. However, damaged arterial surfaces within the vascular system are highly susceptible to thrombus formation. Abnormal platelet deposition, resulting in thrombosis, is more likely to occur in vessels in which endothelial, medial and adventitial damage has occurred. While systemic drugs have been used to prevent coagulation and to inhibit platelet aggregation, a need exists for a means by which a damaged vessel can be treated directly to prevent thrombus formation and subsequent intimal smooth muscle cell proliferation.

Current treatment regimes for stenosis or occluded vessels include mechanical interventions. However, these techniques also serve to exacerbate the injury, precipitating new smooth muscle cell proliferation and neointimal growth. For example, stenotic arteries are often treated with balloon angioplasty, which involves the mechanical dilation of a vessel with an inflatable catheter. The effectiveness of this procedure is limited in some patients because the treatment itself damages the vessel, thereby inducing proliferation of smooth muscle cells and reocclusion or restenosis of the vessel. It has been estimated that approximately 30 to 40 percent of patients treated by balloon angioplasty and/or stents may experience restenosis within one year of the procedure.

To overcome these problems, numerous approaches have been taken to providing stents useful in the repair of damaged vasculature. In one aspect, the stent itself reduces restenosis in a mechanical way by providing a larger lumen. For example, some stents gradually enlarge over time. To prevent damage to the lumen wall during implantation of the stent, many stents are implanted in a contracted form mounted on a partially expanded balloon of a balloon catheter and then expanded in situ to contact the lumen wall. U.S. Pat. No. 5,059,211 discloses an expandable stent for supporting the interior wall of a coronary artery wherein the stent body is made of a porous bioabsorbable material. To aid in avoiding damage to vasculature during implant of such stents, U.S. Pat. No. 5,662,960 discloses a friction-reducing coating of commingled hydrogel suitable for application to polymeric plastic, rubber or metallic substrates that can be applied to the surface of a stent.

A number of agents that affect cell proliferation have been tested as pharmacological treatments for stenosis and restenosis in an attempt to slow or inhibit proliferation of smooth muscle cells. These compositions have included heparin, coumarin, aspirin, fish oils, calcium antagonists, steroids, prostacyclin, ultraviolet irradiation, and others. Such agents may be systemically applied or may be delivered on a more local basis using a drug delivery catheter or a drug eluting stent. In particular, biodegradable polymer matrices loaded with a pharmaceutical may be implanted at a treatment site. As the polymer degrades, a medicament is released directly at the treatment site. The rate at which the drug is delivered is dependent upon the rate at which the polymer matrix is resorbed by the body. U.S. Pat. No. 5,342,348 to Kaplan and U.S. Pat. No. 5,419,760 to Norciso are exemplary of this technology. U.S. Pat. No. 5,766,710 discloses a stent formed of composite biodegradable polymers of different melting temperatures.

Porous stents formed from porous polymers or sintered metal particles or fibers have also been used for release of therapeutic drugs within a damaged vessel, as disclosed in U.S. Pat. No. 5,843,172. However, tissue surrounding a porous stent tends to infiltrate the pores. In certain applications, pores that promote tissue ingrowth are considered to be counterproductive because the growth of neointima can occlude the artery, or other body lumen, into which the stent is being placed.

Delivery of drugs to the damaged arterial wall components has also been explored by using latticed intravascular stents that have been seeded with sheep endothelial cells engineered to secrete a therapeutic protein, such as t-PA (D. A. Dichek et al., Circulation, 80:1347-1353, 1989). However, endothelium is known to be capable of promoting both coagulation and thrombolysis.

Another approach to controlling the healing of a damaged artery or vein is to induce apoptosis in neointimal cells to reduce the size of a stenotic lesion. U.S. Pat. No. 5,776,905 to Gibbons et al., which is incorporated herein by reference in its entirety, describes induction of apoptosis by administering anti-sense oligonucleotides that counteract the anti-apoptotic gene, bcl-x, which is expressed at high levels by neointimal cells. These anti-sense oligonucleotides are intended to block expression of the anti-apoptotic gene bcl-x so that the neointimal cells are induced to undergo programmed cell death.

Under certain conditions, the body naturally produces another drug that has an influence on cell apoptosis among its many effects. As is explained in U.S. Pat. No. 5,759,836 to Amin et al., which is incorporated herein by reference in its entirety, nitric oxide (NO) is produced by an inducible enzyme, nitric oxide synthase, which belongs to a family of proteins beneficial to arterial homeostasis.

However, the effect of nitric oxide in the regulation of apoptosis is complex. A pro-apoptotic effect seems to be linked to pathophysiological conditions wherein high amounts of NO are produced by the inducible nitric oxide synthase. By contrast, an anti-apoptotic effect results from the continuous, low level release of endothelial NO, which inhibits apoptosis and is believed to contribute to the anti-atherosclerotic function of NO. Dimmeler in “Nitric Oxide and Apoptosis: Another Paradigm For The Double-Edged Role of Nitric Oxide” (Nitric Oxide 1(4): 275-281, 1997) discusses the pro- and anti-apoptotic effects of nitric oxide.

To prevent neointimal proliferation that leads to stenosis or restenosis, U.S. Pat. No. 5,766,584 to Edelman et al. describes a method for inhibiting vascular smooth muscle cell proliferation following injury to the endothelial cell lining by creating a matrix containing endothelial cells and surgically wrapping the matrix about the tunica adventitia. The matrix, and especially the endothelial cells attached to the matrix, secrete products that diffuse into surrounding tissue, but do not migrate to the endothelial cell lining of the injured blood vessel.

In a healthy individual in response to endothelial damage, the vascular endothelium participates in many homeostatic mechanisms important for normal wound healing, the regulation of vascular tone and the prevention of thrombosis. A primary mediator of these functions is endothelium-derived relaxing factor (EDRF). First described in 1980 by Furchgott and Zawadzki (Furchgott and Zawadzki, Nature (Lond.) 288:373-376, 1980) EDRF is either nitric oxide (Moncada et al., Pharmacol Rev. 43:109-142, 1991.) (NO) or a closely related NO-containing molecule (Myers et al., Nature (Lond.), 345:161-163, 1990).

Removal or damage to the endothelium is a potent stimulus for neointimal proliferation, a common mechanism underlying the restenosis of atherosclerotic vessels after balloon angioplasty. (Liu et al., Circulation, 79:1374-1387, 1989); (Fems et al., Science, 253:1129-1132, 1991). Stent-induced restenosis is caused by local wounding of the luminal wall of the artery. Further, restenosis is the result of a chronically-stimulated wound-healing cycle.

The natural process of wound healing involves a two-phase cycle: blood coagulation and inflammation at the site of the wound. In healthy individuals, these two cycles are counterbalanced, each including a natural negative feedback mechanism that prevents over-stimulation. For example, in the coagulation enzyme pathway thrombin factor Xa operates upon factor VII to control thrombus formation and, at the same time stimulates production of PARs (Protease Activated Receptors) by pro-inflammatory monocytes and macrophages. Nitric oxide produced endogenously by endothelial cells regulates invasion of the proinflammatory monocytes and macrophages. In the lumen of an artery, this two-phase cycle results in influx and proliferation of healing cells through a break in the endothelium. Stabilization of the vascular smooth muscle cell population by this natural two-phase counterbalanced process is required to prevent neointimal proliferation leading to restenosis. The absence or scarcity of endogenously produced nitric oxide caused by damage to the endothelial layer in the vasculature is thought to be responsible for the proliferation of vascular smooth muscle cells that results in restenosis following vessel injury, for example following angioplasty.

Nitric oxide dilates blood vessels (Valiance et al., Lancet, 2:997-1000, 1989), inhibits platelet activation and adhesion (Radomski et al., Br. J Pharmacol, 92:181-187, 1987) and, in vitro, nitric oxide limits the proliferation of vascular smooth muscle cells (Garg et al., J. Clin. Invest., 83:1774-1777, 1986). Similarly, in animal models, suppression of platelet-derived mitogens by nitric oxide decreases intimal proliferation (Fems et al., Science, 253:1129-1132, 1991). The potential importance of endothelium-derived nitric oxide in the control of arterial remodeling after injury is further supported by recent preliminary reports in humans suggesting that systemic NO donors reduce angiographic-restenosis six months after balloon angioplasty (The ACCORD Study Investigators, J. Am. Coll. Cardiol. 23:59A. (Abstr.), 1994).

Damage to the endothelial and medial layers of a blood vessel, such as often occurs in the course of balloon angioplasty and stent procedures, has been found to stimulate neointimal proliferation, leading to restenosis of atherosclerotic vessels.

The earliest understanding of the function of the endothelium within an artery was its action as a barrier between highly reactive, blood borne materials and the intima of the artery. A wide variety of biological activity within the artery wall is generated when platelets, monocytes and neutrophils infiltrate intima. These reactions result from release of activating factors such as ATP and PDGF from platelets and IL-1, IL-6, TNFa and bFGF from monocytes and neutrophils. An important consequence of release of these activating factors is a change in the cellular structure of smooth muscle cells, causing the cells to shift from quiescent to migratory. This cellular change is of particular importance in vascular medicine, since activation of quiescent smooth muscle cells in arteries can lead to uncontrolled proliferation, leading to the blockage or narrowing of arteries known as stenosis or restenosis.

The standard of care for the non-surgical treatment of blocked arteries is to re-open the blockage with an angioplasty balloon, often followed by the placement of a wire metal structure called a stent to retain the opening in the artery. An unfortunate consequence of this procedure is the nearly total destruction of the endothelial layer by expansion of the angioplasty balloon and precipitation of foreign body inflammatory response to the stent. Therefore, after removal of the balloon catheter used in the angioplasty, the artery is rapidly exposed to an influx of activating factors. Since mechanical intervention has destroyed the natural blood/artery barrier, all too often the result is a local uncontrolled proliferative response by smooth muscle cells leading to restenosis.

Thus, a need exists in the art for new and better methods and devices for stimulating and supplementing endogenous endothelial production of nitric oxide for the prevention of neointimal proliferation in vasculature having damage to the endothelial lining. Particularly, the need exists for better methods and devices for restoring the natural process of wound healing in damaged arteries and other blood vessels.

### SUMMARY OF THE INVENTION

The present invention is based on the discovery that stents can be coated with biodegradable, bioactive polymers that promote endogenous healing processes at a site of stent implantation. The polymers biodegrade over time, releasing bioactive agents which establish or re-establish the natural healing process in an artery. A released bioactive agent can either be absorbed into a target cell where it acts intracellularly, either within the cytosol, the nucleus, or both, or the bioactive agent can bind to a cell surface receptor molecule to elicit a cellular response without entering the cell. Alternatively, the active agent attached to the polymers (e.g., the polymer backbone) promotes endogenous healing processes at the site of stent implantation by contact with the surroundings into which the stent is implanted, e.g., natural or therapeutic blood components, artery wall, and the like. In the latter case, the healing properties of the stent take place even before biodegradation of the stent.

In one embodiment, there are provided bioactive implantable stents including a stent structure with a surface coating of a biodegradable, bioactive polymer with a polymer backbone, and at least one bioactive agent covalently bound to the polymer backbone so that the bioactive agent is produced in situ as a result of biodegradation of the polymer.

In another embodiment, there are provided a bioactive vascular stents including a stent structure with a surface coating of a biodegradable, bioactive polymer, and at least one ligand that attaches to (i.e. captures) progenitor endothelial cells (PECs) is covalently bonded to the polymer. This ligand may itself be bioactive in also activating the PECs, or it may act in conjunction with another bioactive PEC activating agent.

In yet another embodiment, the invention provides bioactive implantable stents having a porous stent structure; and a multilayered tubular coating encapsulating the stent structure. The multilayered tubular coating has at least three layers: 1) an outer drug-eluting biodegradable polymer layer that sequesters an unbound drug; 2) an inner layer of a biodegradable, bioactive polymer with at least one bioactive agent that produces a therapeutic effect in situ covalently bound thereto; and 3) a drug-impermeable biodegradable barrier layer lying between and in contact with the outer layer and the inner layer.

In still another embodiment, the invention provides methods for treating a patient having a vessel with a damaged endothelium by implanting an invention stent in the vessel at the locus of damage and allowing the stent to biodegrade within the vessel.

In yet another embodiment, the invention provides methods for promoting natural healing of an artery having an endothelium damaged by mechanical intervention. In this method, an invention stent is implanted into the artery following the mechanical intervention under conditions suitable for promoting natural healing of the artery.

### BRIEF DESCRIPTION OF THE FIGURES

FIG. 1 is a schematic cross-section of an invention multilayered polymer-coated stent.

FIG. 2 is a graph illustrating the effect of various bioagents used in invention stents (see Table 1) on adhesion and proliferation of endothelial cells growing on gelatin coated surfaces. Control=zero concentration of bioagent.

FIG. 3 is a graph illustrating the effect of various bioagents used in invention stents (see Table 1 on adhesion and proliferation of smooth muscle cells growing on gelatin coated surfaces. Control=zero concentration of bioagent.

### DETAILED DESCRIPTION OF THE INVENTION

This invention provides stents and methods designed to re-establish a blood/artery barrier concurrently with the placement of the stent in a damaged artery. The invention stents comprise a compatible, reabsorbable polymeric sheath that encapsulates the stent structure. In a preferred embodiment of the invention methods, the stent is placed at the conclusion of the angioplasty procedure, or other medical procedure that damages the arterial endothelium, without allowing a lapse of time sufficient for infiltration of inflammatory factors from the blood stream into the artery wall. In this method, the stent is placed at the location of the damage and preferably immediately covers and protects the area of damaged endothelium so as to prevent infiltration of inflammatory factors from the blood stream into the artery wall, thereby limiting the proliferation of smooth muscle cells.

In other words, the invention stents perform as an artificial endothelial layer while promoting the natural cycle of endothelial healing as described herein. The polymeric sheath may have additional features that contribute to the healing of the artery. In one embodiment, the invention sheath or covering comprises multiple layers, each of which can perform a distinct function in re-establishing a stable lesion and contributing to healing of the injured artery wall.

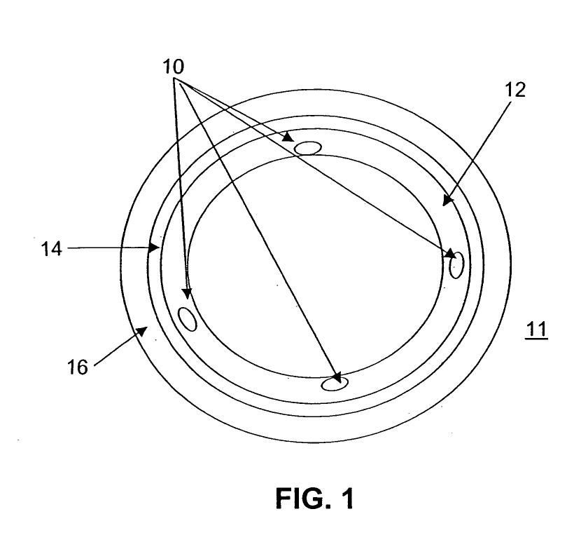

FIG. 1 shows a schematic cross-section of an example of an invention stent 11 with stent struts 10 and a multilayered sheath or covering. When the multilayered stent is implanted, the outer layer 16 of the stent sheath lies directly next to the artery wall. A diffusion barrier layer 14 lies between and is in contact with outer layer 16 and inner layer 12.

The outer layer comprises a polymer layer loaded with a bioactive agent and/or an additional bioactive agent, or combination thereof, specifically including those that limit cellular proliferation or reduce inflammation as disclosed herein. These cellular proliferation limiting and/or inflammation reducing drugs and bioactive agents can be solubilized in the polymer solid phase and, hence are preferably not bound to the polymer of the outer layer, but are loaded into the polymer and sequestered there until the stent is put into place. Once implanted, the active agents in the outer layer 16 diffuse into the artery wall. Preferred additional bioactive agents for incorporation into the outer layer of invention multilayered stents include rapamycin and any of its analogs or derivatives, paclitaxel or any of its analogs or derivatives, everolimus or any of its analogs or derivatives, and statins such as simvastatin. In the outer layer, non-covalently bound bioactive agents and/or additional bioactive agents can be intermingled with or “loaded into” any biocompatible biodegradable polymer as is known in the art since the outer layer in this embodiment of the invention does not come into contact with blood.

Lying along and covering the interior surface of the outer layer of the covering is a diffusion barrier layer 12 of reabsorbable polymer that acts as a diffusion barrier to the drug or biologic contained in the outer layer. The purpose of this diffusion barrier is to direct elution of the drug/biologic into the artery wall to prevent proliferation of smooth muscle cells, while limiting or preventing passage of the drug/biologic into the inner layer. The diffusion barrier layer 12 can accomplish its purpose of partitioning of the drug through hydrophobic/hydrophilic interaction related to the solubility of the bioactive agent in the polymer solid phase. For example, if the bioactive agent or additional bioactive agent in the outer layer is hydrophobic, the polymer barrier layer is selected to be less hydrophobic than the agent(s), and if the bioactive agent or additional bioactive agent in the outer layer is hydrophilic, the barrier layer is selected to be hydrophobic. For example, the barrier layer can be selected from such polymers as polyester, poly(amino acid), poly(ester amide), poly(ester urethane), polyurethane, polylactone, poly(ester ether), or copolymers thereof, whose charge properties are well known by those of skill in the art.

For fabrication of the inner layer 12 of the invention multilayered, bioabsorbable stent, which is exposed to the circulating blood with its endothelial progenitor cells, a blood-compatible, polymer of the type specifically described herein (e.g., having a chemical structure described by structures I through VI herein) is used. One or more bioactive agent, but not “an additional bioactive agent” (i.e., one not involved in the natural processes of endothelialization), is covalently attached to the polymer in the inner layer using techniques described herein. As in other embodiments of the invention stents, the bioactive agent is selected to activate and attract circulating endothelial progenitor cells to the inner layer of the sheath, thereby beginning the process of re-establishing the natural endothelial cell layer.

In one embodiment, the stent structure used in manufacture of the invention multilayered stent is made of a bioabsorbable material with sufficient strength and stiffness to replace a conventional stent, such as a stainless steel or wire mesh stent structure. A cross-linked poly(ester amide), polycaprolactone, or poly(ester urethane) as described herein can be used for this purpose so that the stent is completely bioabsorbable, for example, over a period of three months to twelve months. In this case, over time, each of the layers, and the stent structure as well, will be re-absorbed by the body through natural enzymatic action, allowing the re-established endothelial cell layer to resume its dual function of acting as a blood/artery barrier and providing natural control and stabilization of the intra-cellular matrix within the artery wall through the production of nitric oxide.

In another embodiment, the invention provides bioactive implantable stents including a stent structure with a surface coating of a biodegradable, bioactive polymer, wherein the polymer includes at least one bioactive agent covalently bound to the polymer, and wherein at least one therapeutic bioactive agent is produced in situ as a result of biodegradation of the polymer.

As used herein, “biodegradable” means that at least the polymer coating of the invention stent is capable of being broken down into innocuous and bioactive products in the normal functioning of the body. In a preferred embodiment, the entire stent, including the stent structure is biodegradable. The biodegradable, bioactive polymers have hydrolyzable ester linkages which provide the biodegradability, and are typically chain terminated with carboxyl groups.

Polymers suitable for use in the practice of the invention bear functionalities that allow for facile covalent attachment of bioactive agents to the polymer. For example, a polymer bearing carboxyl groups can readily react with a bioactive agent having an amino moiety, thereby covalently bonding the bioactive agent to the polymer via the resulting amide group. As will be described herein, the biodegradable, bioactive polymer and the bioactive agent can contain numerous complementary functional groups that can be used to covalently attach the bioactive agent to the biodegradable, bioactive polymer.

As used herein, “bioactive” means the polymer plays an active role in the endogenous healing processes at a site of stent implantation by releasing a bioactive or therapeutic agent during biodegradation of the polymer. Bioactive agents contemplated for covalent attachment to the polymers used in coating the invention stents include agents that when freed from the polymer backbone during polymer degradation promote endogenous production of a therapeutic natural wound healing agent, such as nitric oxide endogenously produced by endothelial cells Alternatively the bioactive agents released from the polymers during degradation may be directly active in promoting natural wound healing processes by endothelial cells while controlling proliferation of smooth muscle cells in the vessel at the locus of the damage. These bioactive agents can be any agent that donates, transfers, or releases nitric oxide, elevates endogenous levels of nitric oxide, stimulates endogenous synthesis of nitric oxide, or serves as a substrate for nitric oxide synthase or that inhibits proliferation of smooth muscle cells. Such agents include, for example, aminoxyls, furoxans, nitrosothiols, nitrates and anthocyanins; nucleosides such as adenosine and nucleotides such as adenosine diphosphate (ADP) and adenosine triphosphate (ATP); neurotransmitter/neuromodulators such as acetylcholine and 5-hydroxytryptamine (serotonin/5-HT); histamine and catecholamines such as adrenalin and noradrenalin; lipid molecules such as sphingosine-1-phosphate and lysophosphatidic acid; amino acids such as arginine and lysine; peptides such as the bradykinins, substance P and calcium gene-related peptide (CGRP), and proteins such as insulin, vascular endothelial growth factor (VEGF), and thrombin.

In addition, examples of bioactive agents for the capture of PECs are monoclonal antibodies directed against a known PEC surface marker. Complementary determinants (CDs) that have been reported to decorate the surface of endothelial cells include CD31, CD34+, CD34-, CD102, CD105, CD106, CD109, CDw130, CD141, CD142, CD143, CD144, CDw145, CD146, CD147, and CD166 These cell surface markers can be of varying specificity and the degree of specificity for a particular cell/developmental type/stage is in many cases not fully characterized. In addition these cell marker molecules against which antibodies have been raised will overlap (in terms of antibody recognition) especially with CDs on cells of the same lineage: monocytes in the case of endothelial cells. Circulating endothelial progenitor cells are some way along the developmental pathway from (bone marrow) monocytes to mature endothelial cells. CDs 106, 142 and 144 have been reported to mark mature endothelial cells with some specificity. CD34 is presently known to be specific for progenitor endothelial cells and therefore is currently preferred for capturing progenitor endothelial cells out of blood circulating in the vessel into which the stent is implanted. Examples of such antibodies include single-chain antibodies, chimeric antibodies, monoclonal antibodies, polyclonal antibodies, antibody fragments, Fab fragments, IgA, IgG, IgM, IgD, IgE and humanized antibodies.

Small proteinaceous motifs, such as the B domain of bacterial Protein A and the functionally equivalent region of Protein G, that are known to bind to, and thereby capture such antibody molecules can be attached to the polymer coatings on the stent structure and will act as ligands to capture antibodies by the Fc region out of the patient's blood stream. Therefore, the antibody types that can be attached to polymer coatings using a Protein A or Protein G function region are those that contain an Fc region. The captured antibodies will in turn bind to and hold captured progenitor endothelial cells near the polymer surface while other activating factors, such as the bradykinins, activate the progenitor endothelial cells.

Aminoxyls contemplated for use as bioactive agents have the structure:

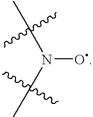

Exemplary aminoxyls include the following compounds:

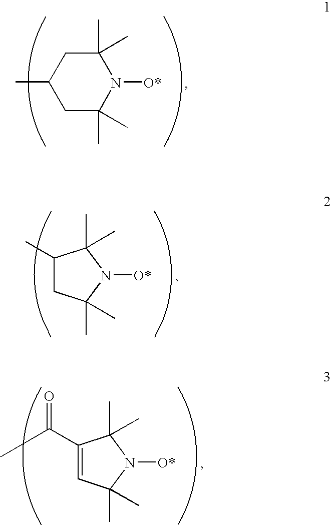

2,2,6,6-tetramethylpiperidine-1-oxy (1); 2,2,5,5-tetramethylpyrrolidine-1-oxy (2); and 2,2,5,5-tetramethylpyrroline-1-oxy-3-carbonyl (3). Further aminoxyls contemplated for use include 4-amino-2,2,6,6-tetramethylpiperidine-1-oxy (TEMPAMINE); 4-(N,N-dimethyl-N-hexadecyl)ammonium-2,2,6,6-tetramethylpiperidine-1-oxy, iodide (CAT16); 4-(N,N-dimethyl-N-(2-hydroxyethyl))ammonium-2,2,6,6-tetramethylpiperidine-1-oxy(TEMPO choline); 4-(N,N-dimethyl-N-(3-sulfopropyl)ammonium-2,2,6,6-tetramethylpiperidine-1-oxy; N-(4-(iodoacetyl)amino-2,2,6,6-tetramethylpiperidine-1-oxy(TEMPO 1A); N-(2,2,6,6-tetramethylpiperidine-1-oxy-4-yl)maleimide(TEMPO maleimide, MAL-6); and 4-trimethylammonium-2,2,6,6-tetramethylpiperidine-1-oxy, iodide (CAT 1); 3-amino-2,2,5,5-tetramethylpyrrolidine-1-oxy; and N-(3-(iodoacetyl)amino)-2,2,5,5-tetramethylpyrrolidine-1-oxy(PROXYL 1A); succinimidyl 2,2,5,5-tetramethyl-3-pyrroline-1-oxy-3-carboxylate and 2,2,5,5-tetramethyl-3-pyrroline-1-oxy-3-carboxylic acid, and the like.

Furoxans contemplated for use as bioactive agents have the structure:

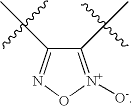

An exemplary furoxan is 4-phenyl-3-furoxancarbonitrile, as set forth below:

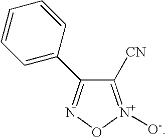

Nitrosothiols include compounds bearing the —S—N═O moiety, such as the exemplary nitrosothiol set forth below:

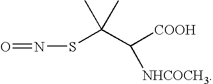

Anthocyanins are also contemplated for use as bioactive agents. Anthocyanins are glycosylated anthocyanidins and have the structure:

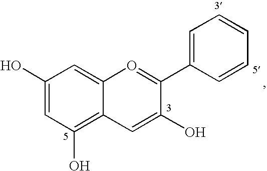

wherein the sugars are attached to the 3-hydroxy position. Anthocyanins are known to stimulate NO production in vivo and therefore are suitable for use as bioactive agents in the practice of the invention.

In further embodiments, the bioactive agent is a ligand for attaching to or capturing progenitor endothelial cells floating within the blood stream within the blood vessel. In one embodiment, the ligand is a “sticky” peptide or polypeptide, such as Protein A and Protein G. Protein A is a constituent of staphylococcus A bacteria that binds the Fc region of particular antibody or immunoglobulin molecules, and is used extensively to identify and isolate these molecules. For example the Protein A ligand can be or contain the amino acid sequence:

| | |
|---|---|
| MTPAVTTYKLVINGKTLKGETTTKAVDAETAEKAFKQYANDNGVDGVWTYDDATKTFTVTE | (SEQ ID NO:1) |

or a functionally equivalent peptidic derivative thereof, such as, by way of an example, the functionally equivalent peptide having the amino acid sequence:

| | |
|---|---|
| TYKLILNGKTLKGETTTEAVDAATAEKVFKQYANDNGVDGEWTYDDATKTFTVTE | (SEQ ID NO:2) |

Protein G is a constituent of group G streptococci bacteria, and displays similar activity to Protein A, namely binding the Fc region of particular antibody or immunoglobulin molecules. For example, the Protein G ligand can be, or contain Protein G having an amino acid sequence:

| | |
|---|---|
| MTPAVTTYKLVINGKTLKGETTTKAVDAETAEKAFKQYANDNGVDGVWTYDDATKTFTVTE | (SEQ ID NO:3) |

or a functionally equivalent peptidic derivative thereof, such as, by way of an example, the functionally equivalent peptide having the amino acid sequence:

| | |
|---|---|
| TYKLILNGKTLKGETTTEAVDAATAEKVFKQYANDNGVDGEWTYDDATKTFTVTE | (SEQ ID NO:4) |

Other bioactive peptides contemplated for attachment to the polymer backbone in the polymer coatings covering the invention medical devices (e.g., surface coatings of stents and sheaths for covering the stent structure) include the bradykinins. Bradykinins are vasoactive nonapeptides formed by the action of proteases on kininogens, to produce the decapeptide kallidin (KRPPGFSPFR) (SEQ ID NO:5), which can undergo further C-terminal proteolytic cleavage to yield the bradykinin 1 nonapeptide: (KRPPGFSPF) (SEQ ID NO: 6), or N-terminal proteolytic cleavage to yield the bradykinin 2 nonapeptide: (RPPGFSPFR) (SEQ ID NO: 7). Bradykinins 1 and 2 are functionally distinct as agonists of specific bradykinin cell surface receptors B 1 and B2 respectively: both kallidin and bradykinin 2 are natural ligands for the B2 receptor whereas their C-terminal metabolites (bradykinin 1 and the octapeptide RPPGFSPF (SEQ ID NO:8) respectively) are ligands for the BI receptor. A portion of circulating bradykinin peptides can be subject to a further post-translational modification: hydroxylation of the second proline residue in the sequence (Pro3 to Hyp3 in the bradykinin 2 amino acid numbering). Bradykinins are very potent vasodilators, increasing permeability of post-capillary venules, and acting on endothelial cells to activate calmodulin and thereby nitric oxide synthase.

Bradykinin peptides are incorporated into the polymers used in the invention stents by attachment at one end of the peptide. The unattached end of the bradykinin extends freely from the polymer to contact endothelial cells in the vessel wall as well as progenitor endothelial cells floating in the blood vessel into which the stent is implanted, thereby activating the endothelial cells with which contact is made. Endothelial cells activated in this way activate further progenitor endothelial cells with which they come into contact, thereby causing a cascade of endothelial cell activation at the site of the injury that results in endogenous production of nitric oxide.

In a still further aspect, the bioactive agent can be a nucleoside, such as adenosine, which is also known to be a potent activator of endothelial cells to produce nitric oxide endogenously.

Polymers contemplated for use in forming the blood-compatible, hydrophilic coating or inner layer in the invention stents include polyesters, poly(amino acids), polyester amides, polyurethanes, or copolymers thereof. In particular, examples of biodegradable polyesters include poly(α-hydroxy C₁-C₅ alkyl carboxylic acids), e.g., polyglycolic acids, poly-L-lactides, and poly-D,L-lactides; poly-3-hydroxy butyrate; polyhydroxyvalerate; polycaprolactones, e.g., poly(α-caprolactone); and modified poly(α-hydroxyacid)homopolymers, e.g., homopolymers of the cyclic diester monomer, 3-(S)[alkyloxycarbonyl)methyl]-1,4-dioxane-2,5-dione which has the formula 4 where R is lower alkyl, depicted in Kimura, Y., “Biocompatible Polymers” in Biomedical Applications of Polymeric Materials, Tsuruta, T., et al, eds., CRC Press, 1993 at page 179.

Examples of biodegradable copolymer polyesters useful in forming the blood-compatible, hydrophilic coating or inner layer in the invention stents include copolyester amides, copolyester urethanes, glycolide-lactide copolymers, glycolide-caprolactone copolymers, poly-3-hydroxy butyrate-valerate copolymers, and copolymers of the cyclic diester monomer, 3-(S)[(alkyloxycarbonyl)methyl]-1,4-dioxane-2,5-dione, with L-lactide. The glycolide-lactide copolymers include poly(glycolide-L-lactide) copolymers formed utilizing a monomer mole ratio of glycolic acid to L-lactic acid ranging from 5:95 to 95:5 and preferably a monomer mole ratio of glycolic acid to L-lactic acid ranging from 45:65 to 95:5. The glycolide-caprolactone copolymers include glycolide and α-caprolactone block copolymer, e.g., Monocryl or Poliglecaprone.

Further examples of polymers contemplated for use in the practice of the invention include those set forth in U.S. Pat. Nos. 5,516,881; 6,338,047; 6,476,204; 6,503,538; and in U.S. application Ser. Nos. 10/096,435; 10/101,408; 10/143,572; and 10/194,965; the entire contents of each of which are incorporated herein by reference.

The biodegradable polymers and copolymers preferably have weight average molecular weights ranging from 10,000 to 125,000; these polymers and copolymers typically have inherent viscosities at 25° C., determined by standard viscosimetric methods, ranging from 0.3 to 4.0, preferably ranging from 0.5 to 3.5. Poly(caprolactones) contemplated for use have an exemplary structure (I) as

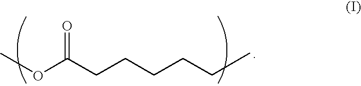

Poly(glycolides) contemplated for use have an exemplary structure (II) as follows:

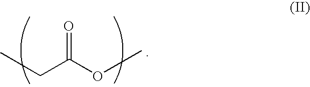

Poly(lactides) contemplated for use have an exemplary structure (III) as follows:

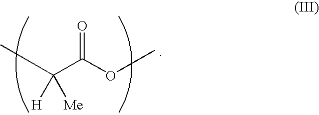

An exemplary synthesis of a suitable poly(lactide-co-ε-caprolactone) including an aminoxyl moiety is set forth as follows. The first step involves the copolymerization of lactide and ε-caprolactone in the presence of benzyl alcohol using stannous octoate as the catalyst to form a polymer of structure (IV).

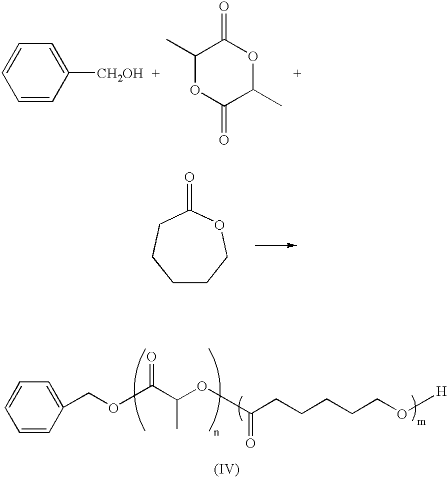

The hydroxy terminated polymer chains can then be capped with maleic anhydride to form polymer chains having structure (V):

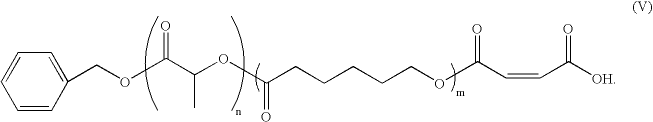

At this point, 4-amino-2,2,6,6-tetramethylpiperidine-1-oxy can be reacted with the carboxylic end group to covalently attach the aminoxyl moiety to the copolymer via the amide bond which results from the reaction between the 4-amino group and the carboxylic acid end group. Alternatively, the maleic acid capped copolymer can be grafted with polyacrylic acid to provide additional carboxylic acid moieties for subsequent attachment of further aminoxyl groups.

Exemplary polyester amides have the structure (VI):

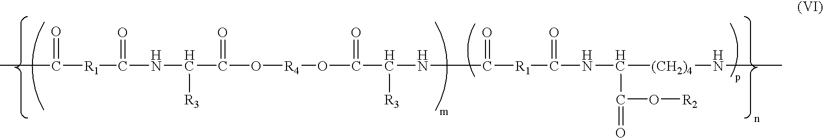

wherein:

m is about 0.1 to about 0.9;

p is about 0.9 to about 0.1;

n is about 50 to about 150;

each R₁ is independently (C₂-C₂₀)alkylene;

each R₂ is independently hydrogen, or (C₆-C₁₀)aryl(C₁-C₆)alkyl;

each R₃ is independently hydrogen, (C₁-C₆)alkyl, (C₂-C₆)alkenyl, (C₂-C₆)alkynyl, or (C₆-C₁₀)aryl(C₁-C₆)alkyl; and

each R₄ is independently (C₂-C₂₀)alkylene.

Polymers contemplated for use in the practice of the invention can be synthesized by a variety of methods well known in the art. For example, tributyltin (IV) catalysts are commonly used to form polyesters such as poly(caprolactone), poly(glycolide), poly(lactide), and the like. However, it is understood that a wide variety of catalysts can be used to form polymers suitable for use in the practice of the invention.

The bioactive agent can be covalently bound to the biodegradable, bioactive polymers via a wide variety of suitable functional groups. For example, when the biodegradable, bioactive polymer is a polyester, the carboxyl group chain end can be used to react with a complimentary moiety on the bioactive agent, such as hydroxy, amino, thio, and the like. A wide variety of suitable reagents and reaction conditions are disclosed, e.g., in Advanced Organic Chemistry, Reactions, Mechanisms, and Structure, Fifth Edition, (2001); and Comprehensive Organic Transformations, Second Edition, Larock (1999).

In other embodiments, a bioactive agent can be linked to any of the polymers of structures (I)-(VI) through an amide, ester, ether, amino, ketone, thioether, sulfinyl, sulfonyl, disulfide, and the like, or a direct linkage. Such a linkage can be formed from suitably functionalized starting materials using synthetic procedures that are known in the art.

In one embodiment of the present invention, a polymer of the present invention can be linked to the bioactive agent via a carboxyl group (e.g., COOH) of the polymer. Specifically, a compound of structures (I)-(VI) can react with an amino functional group of a bioactive agent or a hydroxyl functional group of a bioactive agent to provide a biodegradable, bioactive polymer having a bioactive agent attached via an amide linkage or carboxylic ester linkage, respectively. In another embodiment, the carboxyl group of the polymer can be transformed into an acyl halide, acyl anhydride/“mixed” anhydride, or active ester.

Alternatively, the bioactive agent may be attached to the polymer via a linker. Indeed, to improve surface hydrophobicity of the biodegradable, bioactive polymer, to improve accessibility of the biodegradable, bioactive polymer towards enzyme activation, and to improve the release profile of the biodegradable, bioactive polymer, a linker may be utilized to indirectly attach the bioactive agent to the biodegradable, bioactive polymer. In certain embodiments, the linker compounds include poly(ethylene glycol) having a molecular weight (MW) of about 44 to about 10,000, preferably 44 to 2000; amino acids, such as serine; polypeptides with repeat units from 1 to 100; and any other suitable low molecular weight polymers. The linker typically separates the bioactive agent from the polymer by about 5 angstroms up to about 200 angstroms.

In still further embodiments, the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀) aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

As used herein, the term “alkyl” refers to a straight or branched chain hydrocarbon group including methyl, ethyl, n-propyl, isopropyl, n-butyl, isobutyl, tert-butyl, n-hexyl, and the like.

As used herein, “alkenyl” refers to straight or branched chain hydrocarbyl groups having one or more carbon-carbon double bonds.

As used herein, “alkynyl” refers to straight or branched chain hydrocarbyl groups having at least one carbon-carbon triple bond.

As used herein, “aryl” refers to aromatic groups having in the range of 6 up to 14 carbon atoms.

In certain embodiments, the linker may be a polypeptide having from about 2 up to about 25 amino acids. Suitable peptides contemplated for use include poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, poly-L-lysine-L-tyrosine, and the like.

The linker can be attached first to the polymer or to the bioactive agent. During synthesis of polymers containing bioactive agents indirectly attached via a linker, the linker can either in unprotected form or protected from, using a variety of protecting groups well known to those skilled in the art.

In the case of a protected linker, the unprotected end of the linker can first be attached to the polymer or the bioactive agent. The protecting group can then be de-protected using Pd/H₂ hydrogen lysis, mild acid or base hydrolysis, or any other common de-protection method that are known in the art. The de-protected linker can then be attached to the bioactive agent. An example using poly(ethylene glycol) as the linker is shown in Scheme 1.

Scheme 1

Poly(ethylene glycol) employed as the linker between polymer and drug/biologic.

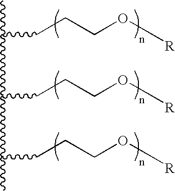

wherein [symbol] represents the polymer;

R can be either a drug or bioactive agent; and

n can range from 1 to 200; preferable from 1 to 50.

An exemplary synthesis of a biodegradable, bioactive polymer according to the invention (wherein the bioactive agent is an aminoxyl) is set forth as follows.

A polyester can be reacted with an aminoxyl, e.g., 4-amino-2,2,6,6-tetramethylpiperidine-1-oxy, in the presence of N,N′-carbonyl diimidazole to replace the hydroxyl moiety in the carboxyl group at the chain end of the polyester with imino linked to aminoxyl-containing radical, so that the imino moiety covalently bonds to the carbon of the carbonyl residue of the carboxyl group. The N,N′-carbonyl diimidazole converts the hydroxyl moiety in the carboxyl group at the chain end of the polyester into an intermediate product moiety which will react with the aminoxyl, e.g., 4-amino-2,2,6,6-tetramethylpiperidine-1-oxy. The aminoxyl reactant is typically used in a mole ratio of reactant to polyester ranging from 1:1 to 100:1. The mole ratio of N,N′-carbonyl diimidazole to aminoxyl is preferably about 1:1.

A typical reaction is as follows. A polyester is dissolved in a reaction solvent and reaction is readily carried out at the temperature utilized for the dissolving. The reaction solvent may be any in which the polyester will dissolve; this information is normally available from the manufacturer of the polyester. When the polyester is a polyglycolic acid or a poly(glycolide-L-lactide) (having a monomer mole ratio of glycolic acid to L-lactic acid greater than 50:50), highly refined (99.9+% pure) dimethyl sulfoxide at 1115° C. to 130° C. or hexafluoroisopropanol at room temperature suitably dissolves the polyester. When the polyester is a poly-L-lactic acid, a poly-DL-lactic acid or a poly(glycolide-L-lactide) (having a monomer mole ratio of glycolic acid to L-lactic acid 50:50 or less than 50:50), tetrahydrofuran, methylene chloride and chloroform at room temperature to 50° C. suitably dissolve the polyester.

The reaction is typically carried out to substantial completion in 30 minutes to 5 hours. When a polyglycolic acid or a poly(glycolide-L-lactide) from a glycol-rich monomer mixture constitutes the polyester, 2 to 3 hours of reaction time is preferred. When a poly-L-lactic acid is the polyester, the reaction is readily carried out to substantial completion at room temperature for one hour. The reaction is preferably carried out under an inert atmosphere with dry nitrogen purging so as to drive the reaction towards completion.

The product may be precipitated from the reaction mixture by adding cold non-solvent for the product. For example, aminoxyl-containing polyglycolic acid and aminoxyl-containing poly(glycolide-L-lactide) formed from glycolic acid-rich monomer mixture are readily precipitated from hot dimethylsulfoxide by adding cold methanol or cold acetone/methanol mixture and then recovered, e.g., by filtering. When the product is not readily precipitated by adding cold non-solvent for the product, the product and solvent may be separated by using vacuum techniques. For example, aminoxyl-containing poly-L-lactic acid is advantageously separated from solvent in this way. The recovered product is readily further purified by washing away water and by-products (e.g. urea) with a solvent which does not dissolve the product, e.g., methanol in the case of the modified polyglycolic acid, polylactic acid and poly(glycolide-L-lactide) products herein. Residual solvent from such washing may be removed using vacuum drying.

Stents according to the invention are typically cylindrical in shape. The walls of the cylindrical structure can be formed of metal or polymer with openings therein, e.g., a mesh. The stent is implanted into a body lumen, such as a blood vessel, where it stays permanently, to keep the vessel open and to improve blood flow to the heart muscle and promote natural would healing processes at a location of damaged endothelium. Stents can also be positioned in vasculature in other parts of the body, such as the kidneys or the brain. The stenting procedure is fairly common, and various types of stents have been developed and used as is known in the art.

The polymers described herein can be coated onto the surface of a porous stent structure or other medical device as described here in many ways, such as dip-coating, spray-coating, ionic deposition, and the like, as is well known in the art. In coating a porous stent, care must be taken not to occlude the pores in the stent structure, which are needed to allow access and migration from the interior of the vessel to the vessel wall of blood borne progenitor endothelial cells and other blood factors that participate in the natural biological process of wound healing.

Alternatively, the polymer coating on the surface of the stent structure can be a formed as a polymer sheath that is applied over the stent structure. In this embodiment the sheath serves as a partial physical barrier to macrophages so that a relatively small number of smooth muscle cells are activated to cause neointimal proliferation. To allow for sufficient movement of bioactive material across the porous stent structure, such as progenitor endothelial cells from the blood stream, the sheath can be laser ablated to form openings in the polymer coating. The stent structure can be moved while the laser is held stationary to ablate the structure into a pattern, or alternatively, the laser can be programmed to move along a predetermined pattern by a method known to artisans. A combination of both, i.e. moving both the laser and the structure, is also possible. In the present invention, even a coated stent having a complex stent pattern can be made with high precision.

The stent structure can be formed of any suitable substance, such as is known in the art, that can be adapted (e.g., molded, stamped, woven, etc.) to contain the porous surface features described herein. For example, the stent body can be formed from a biocompatible metal, such as stainless steel, tantalum, nitinol, elgiloy, and the like, and suitable combinations thereof.

For example, metal stent structures can be formed of a material comprising metallic fibers uniformly laid to form a three-dimensional non-woven matrix and sintered to form a labyrinth structure exhibiting high porosity, typically in a range from about 50 percent to about 85 percent, preferably at least about 70 percent. The metal fibers typically have a diameter in the range from about 1 micron to 25 microns. Pores in the stent structure can have an average diameter in the range from about 30 microns to about 65 microns. For use in coronary arteries, the stent structure should be made of 100% stainless steel, with fully annealed stainless steel being a preferred metal. The stent structure can be of the type that is balloon expandable, as is known in the art.

In one embodiment, the stent structure is itself entirely biodegradable, being made of cross-linkable “star structure polymers”, or dendrimers, which are well known to those skilled in the art. In one aspect, the stent structure is formed from biodegradable cross-linked poly(ester amide), polycaprolactone, or poly(ester urethane) as described herein. In invention multilayered biodegradable stents, the stent structure (i.e., the “stent struts”) is preferably biodegradable and hence are made of such cross-linkable polymers or dendrimers.

Polymer/Bioactive agent Linkage

In one embodiment, the polymers used to make the surface covering for the invention stents and other medical devices as described herein have one or more bioactive agents that promote natural re-endothelialization of vessels directly linked to the polymer. The residues of the polymer can be linked to the residues of the one or more bioactive agents. For example, one residue of the polymer can be directly linked to one residue of the bioactive agent. The polymer and the bioactive agent can each have one open valence. Alternatively, more than one bioactive agent, or a mixture of bioactive agents, that promote natural re-endothelialization of vessels can be directly linked to the polymer. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the linkage that is desired, those skilled in the art can select suitably functionalized starting materials that can be derived from the polymer of the present invention using procedures that are known in the art. As used herein, a “residue of a compound of formula (*)” refers to a radical of a compound of formulas (VI) having one or more open valences. Any synthetically feasible atom, atoms, or functional group of the compound of formulas (I-VI) (e.g., on the polymer backbone or pendant group) can be removed to provide the open valence, provided bioactivity is substantially retained when the radical is attached to a residue of a bioactive agent. Additionally, any synthetically feasible functional group (e.g., carboxyl) can be created on the compound of formulas (I-VI) (e.g., on the polymer backbone or pendant group) to provide the open valance, provided bioactivity is substantially retained when the radical is attached to a residue of a bioactive agent. Based on the linkage that is desired, those skilled in the art can select suitably functionalized starting materials that can be derived from the compound of formulas I-VI) using procedures that are known in the art.

The residue of a bioactive agent can be linked to the residue of a compound of formula (I)-(VI) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) linkage, wherein each R is independently H or (C₁-C₆)alkyl. Such a linkage can be formed from suitably functionalized starting materials using synthetic procedures that are known in the art. Based on the linkage that is desired, those skilled in the art can select suitably functional starting materials that can be derived from a residue of a compound of formula (I)-(VI) and from a given residue of a bioactive agent using procedures that are known in the art. The residue of the bioactive agent can be directly linked to any synthetically feasible position on the residue of a compound of formula (I)-(VI). Additionally, the invention also provides compounds having more than one residue of a bioactive agent or bioactive agents directly linked to a compound of formula (I)-(VI).

One or more bioactive agents can be linked directly to the polymer. Specifically, the residue of each of the bioactive agents can each be directly linked to the residue of the polymer. Any suitable number of bioactive agents (i.e., residues thereof) can be directly linked to the polymer (i.e., residue thereof). The number of bioactive agents that can be directly linked to the polymer can typically depend upon the molecular weight of the polymer. For example, for a compound of formula (VI), wherein n is about 50 to about 150, up to about 450 bioactive agents (i.e., residues thereof) can be directly linked to the polymer (i.e., residue thereof), up to about 300 bioactive agents (i.e., residues thereof) can be directly linked to the polymer (i.e., residue thereof), or up to about 150 bioactive agents (i.e., residues thereof) can be directly linked to the polymer (i.e., residue thereof). Likewise, for a compound of formula (IV), wherein n is about 50 to about 150, up to about 450 bioactive agents (i.e., residues thereof) can be directly linked to the polymer (i.e., residue thereof), up to about 300 bioactive agents (i.e., residues thereof) can be directly linked to the polymer (i.e., residue thereof), or up to about 150 bioactive agents (i.e., residues thereof) can be directly linked to the polymer (i.e., residue thereof).

The residue of a polymer of the present invention, the residue of a compound of formula (VI), and/or the residue of a compound of formula (IV) can be formed employing any suitable reagents and reaction conditions. Suitable reagents and reaction conditions are disclosed, e.g., in Advanced Organic Chemistry, Part B: Reactions and Synthesis, Second Edition, Carey and Sundberg (1983); Advanced Organic Chemistry, Reactions, Mechanisms, and Structure, Second Edition, March (1977); and Comprehensive Organic Transformations, Second Edition, Larock (1999).

In one embodiment of the present invention, a polymer (i.e., residue thereof) can be linked to the bioactive agent (i.e., residue thereof) via the carboxyl group (e.g., COOR²) of the polymer. Specifically, a compound of formula (VI) wherein R² is independently hydrogen, or (C₆-C₁₀)aryl (C₁-C₆)alkyl; can react with an amino functional group of the bioactive agent or a hydroxyl functional group of the bioactive agent, to provide a Polymer/Bioactive agent having an amide linkage or a Polymer/Bioactive agent having a carboxylic ester linkage, respectively. In another embodiment, the carboxyl group of the polymer can be transformed into an acyl halide or an acyl anhydride.

Additional Bioactive Agent

As used herein, an “additional bioactive agent” refers to a therapeutic or diagnostic agent other than the “bioactive” agents described above that promote the natural wound healing process of re-endothelialization of vessels as disclosed herein. Such additional bioactive agents can also be attached polymer coatings on the surface of the invention stents or to polymers used for coating other types of insertable or implantable medical or therapeutic devices having different treatment aims as are known in the art, wherein contact of the polymer coating with a treatment surface or blood borne cell or factor or release from the polymer coating by biodegradation is desirable. However, such additional bioactive agents are not used in the inner layer of the invention multilayered stents, which contain only the bioactive agents that promote the natural would healing process of re-endothelialization of vessels.

Specifically, such additional bioactive agent can include, but is not limited to, one or more: polynucleotides, polypeptides, oligonucleotides, gene therapy agents, nucleotide analogs, nucleoside analogs, polynucleic acid decoys, therapeutic antibodies, abciximab, anti-inflammatory agents, blood modifiers, anti-platelet agents, anti-coagulation agents, immune suppressive agents, anti-neoplastic agents, anti-cancer agents, anti-cell proliferation agents, and nitric oxide releasing agents.

The polynucleotide can include deoxyribonucleic acid (DNA), ribonucleic acid (RNA), double stranded DNA, double stranded RNA, duplex DNA/RNA, antisense polynucleotides, functional RNA or a combination thereof. In one embodiment, the polynucleotide can be RNA. In another embodiment, the polynucleotide can be DNA. In another embodiment, the polynucleotide can be an antisense polynucleotide. In another embodiment, the polynucleotide can be a sense polynucleotide. In another embodiment, the polynucleotide can include at least one nucleotide analog. In another embodiment, the polynucleotide can include a phosphodiester linked 3′-5′ and 5′-3′ polynucleotide backbone. Alternatively, the polynucleotide can include non-phosphodiester linkages, such as phosphotioate type, phosphoramidate and peptide-nucleotide backbones. In another embodiment, moieties can be linked to the backbone sugars of the polynucleotide. Methods of creating such linkages are well known to those of skill in the art.

The polynucleotide can be a single-stranded polynucleotide or a double-stranded polynucleotide. The polynucleotide can have any suitable length. Specifically, the polynucleotide can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense polynucleotide is typically a polynucleotide that is complimentary to an mRNA, which encodes a target protein. For example, the mRNA can encode a cancer promoting protein i.e., the product of an oncogene. The antisense polynucleotide is complimentary to the single-stranded mRNA and will form a duplex and thereby inhibit expression of the target gene, i.e., will inhibit expression of the oncogene. The antisense polynucleotides of the invention can form a duplex with the mRNA encoding a target protein and will disallow expression of the target protein.

A “functional RNA” refers to a ribozyme or other RNA that is not translated.

A “polynucleic acid decoy” is a polynucleic acid which inhibits the activity of a cellular factor upon binding of the cellular factor to the polynucleic acid decoy. The polynucleic acid decoy contains the binding site for the cellular factor. Examples of cellular factors include, but are not limited to, transcription factors, polymerases and ribosomes. An example of a polynucleic acid decoy for use as a transcription factor decoy will be a double-stranded polynucleic acid containing the binding site for the transcription factor. Alternatively, the polynucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such polynucleic acid decoys and target proteins include, but are not limited to, promoter sequences for inhibiting polymerases and ribosome binding sequences for inhibiting ribosomes. It is understood that the invention includes polynucleic acid decoys constructed to inhibit any target cellular factor.

A “gene therapy agent” refers to an agent that causes expression of a gene product in a target cell through introduction of a gene into the target cell followed by expression. An example of such a gene therapy agent would be a genetic construct that causes expression of a protein, such as insulin, when introduced into a cell. Alternatively, a gene therapy agent can decrease expression of a gene in a target cell. An example of such a gene therapy agent would be the introduction of a polynucleic acid segment into a cell that would integrate into a target gene and disrupt expression of the gene. Examples of such agents include viruses and polynucleotides that are able to disrupt a gene through homologous recombination. Methods of introducing and disrupting genes with cells are well known to those of skill in the art.

An oligonucleotide of the invention can have any suitable length. Specifically, the oligonucleotide can be about 2 to about 100 nucleotides in length, inclusive; up to about 20 nucleotides in length, inclusive; or about 15 to about 30 nucleotides in length, inclusive. The oligonucleotide can be single-stranded or double-stranded. In one embodiment, the oligonucleotide can be single-stranded. The oligonucleotide can be DNA or RNA. In one embodiment, the oligonucleotide can be DNA. In one embodiment, the oligonucleotide can be synthesized according to commonly known chemical methods. In another embodiment, the oligonucleotide can be obtained from a commercial supplier. The oligonucleotide can include, but is not limited to, at least one nucleotide analog, such as bromo derivatives, azido derivatives, fluorescent derivatives or a combination thereof. Nucleotide analogs are well known to those of skill in the art. The oligonucleotide can include a chain terminator. The oligonucleotide can also be used, e.g., as a cross-linking reagent or a fluorescent tag. Many common linkages can be employed to couple an oligonucleotide to another moiety, e.g., phosphate, hydroxyl, etc. Additionally, a moiety may be linked to the oligonucleotide through a nucleotide analog incorporated into the oligonucleotide. In another embodiment, the oligonucleotide can include a phosphodiester linked 3′-5′ and 5′-3′ oligonucleotide backbone. Alternatively, the oligonucleotide can include non-phosphodiester linkages, such as phosphotioate type, phosphoramidate and peptide-nucleotide backbones. In another embodiment, moieties can be linked to the backbone sugars of the oligonucleotide. Methods of creating such linkages are well known to those of skill in the art.

Nucleotide and nucleoside analogues are well known on the art. Examples of such nucleoside analogs include, but are not limited to, Cytovene® (Roche Laboratories), Epivir® (Glaxo Wellcome), Gemzar® (Lilly), Hivid® (Roche Laboratories), Rebetron® (Schering), Videx® (Bristol-Myers Squibb), Zerit® (Bristol-Myers Squibb), and Zovirax® (Glaxo Wellcome). See, Physician's Desk Reference, 2001 Edition.

Polypeptides acting as additional bioactive agents attached to the polymers in the invention stent coverings and other medical devices can have any suitable length. Specifically, the polypeptides can be about 2 to about 5,000 amino acids in length, inclusive; about 2 to about 2,000 amino acids in length, inclusive; about 2 to about 1,000 amino acids in length, inclusive; or about 2 to about 100 amino acids in length, inclusive.

The polypeptides can also include “peptide mimetics.” Peptide analogs are commonly used in the pharmaceutical industry as non-peptide bioactive agents with properties analogous to those of the template peptide. These types of non-peptide compound are termed “peptide mimetics” or “peptidomimetics.” Fauchere, J. (1986) Adv. Bioactive agent Res., 15:29; Veber and Freidinger (1985) TINS p. 392; and Evans et al. (1987) J. Med. Cherm., 30:1229; and are usually developed with the aid of computerized molecular modeling. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH₂NH—, —CH₂S—, CH₂—CH₂—, —CH═CH—(cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂ NH—, CH₂CH₂—); Spatola, A. F. et al., Life Sci., (1986) 38:1243-1249 (—CH—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a polypeptide with a D-Lysine in place of L-lysine) may be used to generate more stable polypeptides and polypeptides resistant to endogenous proteases.

In one embodiment, the additional bioactive agent polypeptide attached to the polymer coatings for the invention medical devices can be an antibody. In one embodiment, the antibody can bind to a cell adhesion molecule, such as a cadherin, integrin or selectin. In another embodiment, the antibody can bind to an extracellular matrix molecule, such as collagen, elastin, fibronectin or laminin. In still another embodiment, the antibody can bind to a receptor, such as an adrenergic receptor, B-cell receptor, complement receptor, cholinergic receptor, estrogen receptor, insulin receptor, low-density lipoprotein receptor, growth factor receptor or T-cell receptor. Antibodies attached to polymers (either directly or by a linker in the invention medical devices can also bind to platelet aggregation factors (e.g., fibrinogen), cell proliferation factors (e.g., growth factors and cytokines), and blood clotting factors (e.g., fibrinogen). In another embodiment, an antibody can be conjugated to an active agent, such as a toxin. In another embodiment, the antibody can be Abciximab (ReoProR)). Abciximab is an Fab fragment of a chimeric antibody that binds to beta(3) integrins. Abciximab is specific for platelet glycoprotein IIb/IIIa receptors, e.g., on blood cells. Human aortic smooth muscle cells express alpha(v)beta(3) integrins on their surface. Treating beta(3) expressing smooth muscle cells may prohibit adhesion of other cells and decrease cellular migration or proliferation, thus reducing restenosis following percutaneous coronary interventions (CPI) e.g., stenosis, angioplasty, stenting. Abciximab also inhibits aggregation of blood platelets.

In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin. Examples of glycopeptides included in this definition may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc., 1996, 118, 13107-13108; J. Amer. Chem. Soc., 1997, 119, 12041-12047; and J. Amer. Chem. Soc., 1994, 116, 45734590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics which have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur and phosphorous. Lipidated glycopeptide antibiotics are well-known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory agents useful for attachment to polymer coatings of the invention stents and other medical devices, or for loading into the outer layer of the invention multilayered stents include, e.g. analgesics (e.g., NSAIDS and salicyclates), antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2001 Edition. Specifically, the anti-inflammatory agent can include dexamethasone, which is chemically designated as (11β, 16α)-9-fluro-11,17,21-trihydroxy-16-methylpregna-1,4-diene-3,20-dione. Alternatively, the anti-inflammatory agent can include sirolimus (rapamycin), which is a triene macrolide antibiotic isolated from Steptomyces hygroscopicus.

Anti-platelet or anti-coagulation agents include, e.g., Coumadin®) (DuPont), Fragmin® (Pharmacia & Upjohn), Heparin® (Wyeth-Ayerst), Lovenox®, Normiflo®, Orgaran® (Organon), Aggrastat® (Merck), Agrylin® (Roberts), Ecotrin® (Smithkline Beecham), Flolan® (Glaxo Wellcome), Halfprin® (Kramer), Integrillin® (COR Therapeutics), Integrillin® (Key), Persantine® (Boehringer Ingelheim), Plavix® (Bristol-Myers Squibb), ReoPro® (Centecor), Ticlid® (Roche), Abbokinase® (Abbott), Activase® (Genentech), Eminase® (Roberts), and Strepase® (Astra). See, Physician's Desk Reference, 2001 Edition. Specifically, the anti-platelet or anti-coagulation agent can include trapidil (avantrin), cilostazol, heparin, hirudin, or ilprost.

Trapidil is chemically designated as N,N-dimethyl-5-methyl-[1,2,4]triazolo[1,-5-a]pyrimidin-7-amine.

Cilostazol is chemically designated as 6-[4-(1-cyclohexyl-1H-tetrazol-5-yl)-butoxy]-3,4-dihydro-2(1H)-quinolinone.

Heparin is a glycosaminoglycan with anticoagulant activity; a heterogeneous mixture of variably sulfonated polysaccharide chains composed of repeating units of D-glucosamine and either L-iduronic or D-glucuronic acids.

Hirudin is an anticoagulant protein extracted from leeches, e.g., Hirudo medicinalis.

Iloprost is chemically designated as 5-[Hexahydro-5-hydroxy-4-(3-hydroxy-4-methyl-1-octen-6-ynyl)-2(1H)-pentalenylidene]pentanoic acid.

The immune suppressive agent can include, e.g., Azathioprine® (Roxane), BayRho-D® (Bayer Biological), CellCept® (Roche Laboratories), Imuran® (Glaxo Wellcome), MiCRhoGAM® (Ortho-Clinical Diagnostics), Neoran® (Novartis), Orthoclone OKT3® (Ortho Biotech), Prograf® (Fujisawa), PhoGAM® (Ortho-Clinical Diagnostics), Sandimmune® (Novartis), Simulect® (Novartis), and Zenapax®) (Roche Laboratories).

Specifically, the immune suppressive agent can include rapamycin or thalidomide. Rapamycin is a triene macrolide isolated from Streptomyces hygroscopicus.

Thalidomide is chemically designated as 2-(2,6-dioxo-3-piperidinyl)-1H-iso-indole-1,3(2H)-dione.

Anti-cancer or anti-cell proliferation agents that can be used as an additional bioactive agent, for example, in the outer layer of the invention multilayered stents include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hormone (GNRH) analogues, progestrins, immunomodulators, miscellaneous antineoplastics, photosensitizing agents, and skin and mucous membrane agents. See, Physician's Desk Reference, 2001 Edition.

Suitable adjunct antineoplastic agents include Anzemet® (Hoeschst Marion Roussel), Aredia® (Novartis), Didronel® (MGI), Diflucan® (Pfizer), Epogen® (Amgen), Ergamisol® (Janssen), Ethyol® (Alza), Kytril®) (SmithKline Beecham), Leucovorin® (Immunex), Leucovorin® (Glaxo Wellcome), Leucovorin® (Astra), Leukine® (Immunex), Marinol® (Roxane), Mesnex® (Bristol-Myers Squibb Oncology/Immunology), Neupogen (Amgen), Procrit® (Ortho Biotech), Salagen® (MGI), Sandostatin® (Novartis), Zinecard® (Pharmacia and Upjohn), Zofran® (Glaxo Wellcome) and Zyloprim®) (Glaxo Wellcome).

Suitable miscellaneous alkylating agents include Myleran® (Glaxo Wellcome), Paraplatin® (Bristol-Myers Squibb Oncology/Immunology), Platinol® (Bristol-Myers Squibb Oncology/Immunology) and Thioplex® (Immunex).

Suitable nitrogen mustards include Alkeran® (Glaxo Wellcome), Cytoxan® (Bristol-Myers Squibb Oncology/Immunology), Ifex® (Bristol-Myers Squibb Oncology/immunology), Leukeran® (Glaxo Wellcome) and Mustargen® (Merck).

Suitable nitrosoureas include BiCNU® (Bristol-Myers Squibb Oncology/Immunology), CeeNU®) (Bristol-Myers Squibb Oncology/Immunology), Gliadel® (Rhone-Poulenc Rover) and Zanosar® (Pharmacia and Upjohn).

Suitable antibiotics include Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin®) PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology).

Suitable antimetabolites include Cytostar-U® (Pharmacia and Upjohn), Fludara® (Berlex), Sterile FUDR® (Roche Laboratories), Leustatin® (Ortho Biotech), Methotrexate® (Immunex), Parinethol® (Glaxo Wellcome), Thioguanine® (Glaxo Wellcome) and Xeloda® (Roche Laboratories).

Suitable androgens include Nilandron® (Hoechst Marion Roussel) and Teslac®(Bristol-Myers Squibb Oncology/Immunology).

Suitable antiandrogens include Casodex® (Zeneca) and Eulexin® (Schering).

Suitable antiestrogens include Arimidex® (Zeneca), Fareston®) (Schering), Femara® (Novartis) and Nolvadex® (Zeneca).

Suitable estrogen and nitrogen mustard combinations include Emcyt® (Pharmacia and Upjohn).

Suitable estrogens include Estrace® (Bristol-Myers Squibb) and Estrab®(Solvay).

Suitable gonadotropin releasing hormone (GNRH) analogues include Leupron Depot® (TAP) and Zoladex® (Zeneca).

Suitable progestins include Depo-Provera® (Pharmacia and Upjohn) and Megace® (Bristol-Myers Squibb Oncology/Immunology).

Suitable immunomodulators include Erganisol® (Janssen) and Proleukine (Chiron Corporation).

Suitable miscellaneous antineoplastics include Camptosar® (Pharmacia and Upjohn), Celestone® (Schering), DTIC-Dome® (Bayer), Elspar® (Merck), Etopophos® (Bristol-Myers Squibb Oncology/Immunology), Etopoxide® (Astra), Gemzar® (Lilly), Hexalen® (U.S. Bioscience), Hycantin® (SmithKline Beecham), Hydrea® (Bristol-Myers Squibb Oncology/Immunology), Hydroxyurea® (Roxane), Intron A® (Schering), Lysodren® (Bristol-Myers Squibb Oncology/Immunology), Navelbine® (Glaxo Wellcome), Oncaspar® (Rhone-Poulenc Rover), Oncovin® (Lilly), Proleukin® (Chiron Corporation), Rituxan® (IDEC), Rituxan® (Genentech), Roferon-A® (Roche Laboratories), Taxol® (paclitaxol/paclitaxel, Bristol-Myers Squibb Oncology/Immunology), Taxotere® (Rhone-Poulenc Rover), TheraCys® (Pasteur Merieux Connaught), Tice BCG® (Organon), Velban® (Lilly), VePesid® (Bristol-Myers Squibb Oncology/Immunology), Vesanoid® (Roche Laboratories) and Vumon® (Bristol-Myers Squibb Oncology/Immunology).

Suitable photosensitizing agents include Photofrin® (Sanofi).

Specifically, the anti-cancer or anti-cell proliferation agent can include Taxol® (paclitaxol), a nitric oxide-like compound, or NicOX (NCX-4016). Taxol® (paclitaxol) is chemically designated as 5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine.

A nitric oxide-like compound includes any compound (e.g., polymer) to which is bound a nitric oxide releasing functional group. Suitable nitric oxide-like compounds are S-nitrosothiol derivative (adduct) of bovine or human serum albumin and as disclosed, e.g., in U.S. Pat. No. 5,650,447. See, e.g., Inhibition of neointimal proliferation in rabbits after vascular injury by a single treatment with a protein adduct of nitric oxide; David Marks et al., J. Clin. Invest. (1995); 96:2630-2638. NCX-4016 is chemically designated as 2-acetoxybenzoate 2-(nitroxymethyl)-phenyl ester, and is an antithrombotic agent.

It is appreciated that those skilled in the art understand that the bioactive agent useful in the present invention is the bioactive substance present in any of the bioactive agents or agents disclosed above. For example, Taxol®) is typically available as an injectable, slightly yellow viscous solution. The bioactive agent, however, is a crystalline powder with the chemical name 5β,20-Epoxy-1,2α,4,7β,10β,13α-hexahydroxytax-11-en-9-one 4,10-diacetate 2-benzoate 13-ester with (2R,3S)-N-benzoyl-3-phenylisoserine. Physician's Desk Reference (PDR), Medical Economics Company (Montvale, N.J.), (53rd Ed.), pp. 1059-1067.

As used herein a “residue of a bioactive agent” or “residue of an additional bioactive agent” is a radical of such bioactive agent as disclosed herein having one or more open valences. Any synthetically feasible atom or atoms of the bioactive agent can be removed to provide the open valence, provided bioactivity is substantially retained when the radical is attached to a residue of compound of formula (I) or (VI). Based on the linkage that is desired, those skilled in the art can select suitably functionalized starting materials that can be derived from a bioactive agent using procedures that are known in the art.

The residue of a bioactive agent can be formed employing any suitable reagents and reaction conditions. Suitable reagents and reaction conditions are disclosed, e.g., in Advanced Organic Chemistry, Part B: Reactions and Synthesis, Second Edition, Carey and Sundberg (1983); Advanced Organic Chemistry, Reactions, Mechanisms and Structure, Second Edition, March (1977); and Comprehensive Organic Transformations, Second Edition, Larock (1999).

In certain embodiments, the polymer/bioactive agent linkage can degrade to provide a suitable and effective amount of free bioactive agent. As will be appreciated by those of skill in the art, depending upon the chemical and therapeutic properties of the biological agent, in certain other embodiments, the bioactive agent attached to the polymer performs its therapeutic effect while still attached to the polymer, such as is the case with the “sticky” polypeptides Protein A and Protein G, known herein as “ligands”, which function while attached to the polymer to hold a target molecule close to the polymer, and the bradykinins and antibodies, which function by contacting (e.g., bumping into) a receptor on a target molecule. Any suitable and effective amount of bioactive agent can be released and will typically depend, e.g., on the specific polymer, bioactive agent, and polymer/bioactive agent linkage chosen. Typically, up to about 100% of the bioactive agent can be released from the polymer by degradation of the polymer/bioactive agent linkage. Specifically, up to about 90%, up to 75%, up to 50%, or up to 25% of the bioactive agent can be released from the polymer. Factors that typically affect the amount of the bioactive agent that is released from the polymer is the type of polymer/bioactive agent linkage, and the nature and amount of additional substances present in the formulation.

The polymer/bioactive agent linkage can degrade over a period of time to provide time release of a suitable and effective amount of bioactive agent. Any suitable and effective period of time can be chosen. Typically, the suitable and effective amount of bioactive agent can be released in about twenty-four hours, in about seven days, in about thirty days, in about ninety days, or in about one hundred and twenty days. Factors that typically affect the length of time in which the bioactive agent is released from the polymer/bioactive agent include, e.g., the nature and amount of polymer, the nature and amount of bioactive agent, the nature of the polymer/bioactive agent linkage, and the nature and amount of additional substances present in the formulation.

Polymer/Linker/Bioactive Agent Linkage

In addition to being directly linked to the residue of a compound of formula (I) (VI), the residue of a bioactive agent can also be linked to the residue of a compound of formula (I)-(VI) by a suitable linker. The structure of the linker is not crucial, provided the resulting compound of the invention has an effective therapeutic index as a bioactive agent.

Suitable linkers include linkers that separate the residue of a compound of formula (I)-(VI) from the residue of a bioactive agent by a distance of about 5 angstroms to about 200 angstroms, inclusive. Other suitable linkers include linkers that separate the residue of a compound of formula (I)-(VI) and the residue of a bioactive agent by a distance of about 5 angstroms to about 100 angstroms, inclusive, as well as linkers that separate the residue of a compound of formula (I)-(VI) from the residue of a bioactive agent by a distance of about 5 angstroms to about 50 angstroms, or by about 5 angstroms to about 25 angstroms, inclusive.

The linker can be linked to any synthetically feasible position on the residue of a compound of formula (I)-(VI). Based on the linkage that is desired, those skilled in the art can select suitably functionalized starting materials that can be derived from a compound of formula (I)-(VI) and a bioactive agent using procedures that are known in the art.

The linker can conveniently be linked to the residue of a compound of formula (I)-(VI) or to the residue of a bioactive agent through an amide (e.g., —N(R)C(═O)— or —C(═O)N(R)—), ester (e.g., —OC(═O)— or —C(—O)O—), ether (e.g., —O—), ketone (e.g., —C(═O)—) thioether (e.g., —S—), sulfinyl (e.g., —S(O)—), sulfonyl (e.g., —S(O)₂—), disulfide (e.g., —S—S—), amino (e.g., —N(R)—) or a direct (e.g., C—C) linkage, wherein each R is independently H or (C₁-C₆)alkyl. The linkage can be formed from suitably functionalized starting materials using synthetic procedures that are known in the art. Based on the linkage that is desired, those skilled in the art can select suitably functionalized starting materials that can be derived from a residue of a compound of formula (I)-(VI), a residue of a bioactive agent, and from a given linker using procedures that are known in the art.

Specifically, the linker can be a divalent radical of the formula W-A-Q wherein A is (C₁-C₂₄)alkyl, (C₂-C₂₄)alkenyl, (C₂-C₂₄)alkynyl, (C₃-C₈)cycloalkyl, or (C₆-C₁₀)aryl, wherein W and Q are each independently —N(R)C(═O)—, —C(═O)N(R)—, OC(═O)—, —C(═O)O—, —O—, —S—, —S(O)—, —S(O)₂—, —S—S—, —N(R)—, —C(═O)—, or a direct bond (i.e., W and/or Q is absent); wherein each R is independently H or (C₁-C₆)alkyl.

Specifically, the linker can be a divalent radical of the formula W—(CH₂)ₙ-Q, wherein n is from about 1 to about 20, from about 1 to about 15, from about 2 to about 10, from about 2 to about 6, or from about 4 to about 6; wherein W and Q are each independently —N(R)C(═O)—, —C(═O)N(R)—, —OC(═O)—, —C(═O)O—, —O—, —S—, —S(O)—, —S(O)₂—, —S—S—, —C(═O)—, —N(R)—, or a direct bond (i.e., W and/or Q is absent); wherein each R is independently H or (C₁-C₆)alkyl.

Specifically, W and Q can each independently be —N(R)C(═O)—, —C(═O)N(R)—, —OC(═O)—, —N(R)—, —C(═O)O—, —O—, or a direct bond (i.e., W and/or Q is absent).

Specifically, the linker can be a divalent radical formed from a saccharide.

Specifically, the linker can be a divalent radical formed from a cyclodextrin.

Specifically, the linker can be a divalent radical, i.e., divalent radicals formed from a peptide or an amino acid. The peptide can comprise 2 to about 25 amino acids, 2 to about 15 amino acids, or 2 to about 12 amino acids.

Specifically, the peptide can be poly-L-lysine (i.e., [—NHCH[(CH₂)₄NH₂]CO—]ₘ-Q wherein Q is H, (Ci-C₁₋₄)alkyl, or a suitable carboxy protecting group; and wherein m is about 2 to about 25. The poly-L-lysine can contain about 5 to about 15 residues (i.e., m is from about 5 to about 15). For example, the poly-L-lysine can contain from about 8 to about 11 residues (i.e., m is from about 8 to about 11).

Specifically, the peptide can also be poly-L-glutamic acid, poly-L-aspartic acid, poly-L-histidine, poly-L-ornithine, poly-L-serine, poly-L-threonine, poly-L-tyrosine, poly-L-leucine, poly-L-lysine-L-phenylalanine, poly-L-arginine, or poly-L-lysine-L-tyrosine.

Specifically, the linker can be prepared from 1,6-diaminohexane H₂N(CH₂)₆NH₂, 1,5-diaminopentane H₂N(CH₂)₅NH₂, 1,4-diaminobutane H₂N(CH₂)₄NH₂, or 1,3-diaminopropane H₂N(CH₂)₃NH₂.

One or more bioactive agents can be linked to the polymer through a linker. Specifically, the residue of each of the bioactive agents can each be linked to the residue of the polymer through a linker. Any suitable number of bioactive agents (i.e., residues thereof) can be linked to the polymer (i.e., residue thereof) through a linker. The number of bioactive agents that can be linked to the polymer through a linker can typically depend upon the molecular weight of the polymer. For example, for a compound of formula (VI), wherein n is about 50 to about 150, up to about 450 bioactive agents (i.e., residues thereof) can be linked to the polymer (i.e., residue thereof) through a linker, up to about 300 bioactive agents (i.e., residues thereof) can be linked to the polymer (i.e., residue thereof) through a linker, or up to about 150 bioactive agents (i.e., residues thereof) can be linked to the polymer (i.e., residue thereof) through a linker. Likewise, for a compound of formula (IV), wherein n is about 50 to about 150, up to about 10 to about 450 bioactive agents (i.e., residues thereof) can be linked to the polymer (i.e., residue thereof) through a linker, up to about 300 bioactive agents (i.e., residues thereof) can be linked to the polymer (i.e., residue thereof) through a linker, or up to about 150 bioactive agents (i.e., residues thereof) can be linked to the polymer (i.e., residue thereof) through a linker.

In one embodiment of the present invention, a polymer (i.e., residue thereof) as disclosed herein can be linked to the linker via a carboxyl group (e.g., COOR²) of the polymer.

For example, a compound of formula (VI), wherein R² is independently hydrogen, or (C₆-C₁₀)aryl(C₁-C₆)alkyl, can react with an amino functional group of the linker or a hydroxyl functional group of the linker, to provide a Polymer/Linker having an amide linkage or a Polymer/Linker having a carboxyl ester linkage, respectively. In another embodiment, the carboxyl group can be transformed into an acyl halide or an acyl anhydride.

In one embodiment of the invention, a bioactive agent (i.e., residue thereof) can be linked to the linker via a carboxyl group (e.g., COOR, wherein R is hydrogen, (C₆-C₁₀)aryl(C₁-C₆)alkyl or (C₁-C₆)alkyl) of the linker. Specifically, an amino functional group of the bioactive agent or a hydroxyl functional group of the bioactive agent can react with the carboxyl group of the linker, to provide a Linker/Bioactive agent having an amide linkage or a Linker/Bioactive agent having a carboxylic ester linkage, respectively. In another embodiment, the carboxyl group of the linker can be transformed into an acyl halide or an acyl anhydride.

The polymer/linker/bioactive agent linkage can degrade to provide a suitable and effective amount of bioactive agent. Any suitable and effective amount of bioactive agent can be released and will typically depend, e.g., on the specific polymer, bioactive agent, linker, and polymer/linker/bioactive agent linkage chosen. Typically, up to about 100% of the bioactive agent can be released from the polymer/linker/bioactive agent. Specifically, up to about 90%, up 75%, up to 50%, or up to 25% of the bioactive agent can be released from the polymer/linker/bioactive agent. Factors that typically affect the amount of the bioactive agent released from the polymer/linker/bioactive agent include, e.g., the nature and amount of polymer, the nature and amount of bioactive agent, the nature and amount of linker, the nature of the polymer/linker/bioactive agent linkage, and the nature and amount of additional substances present in the formulation.

The polymer/linker/bioactive agent linkage can degrade over a period of time to provide the suitable and effective amount of bioactive agent. Any suitable and effective period of time can be chosen. Typically, the suitable and effective amount of bioactive agent can be released in about twenty-four hours, in about seven days, in about thirty days, in about ninety days, or in about one hundred and twenty days. Factors that typically affect the length of time in which the bioactive agent is released from the polymer/linker/bioactive agent include, e.g., the nature and amount of polymer, the nature and amount of bioactive agent, the nature of the linker, the nature of the polymer/linker/bioactive agent linkage, and the nature and amount of additional substances present in the formulation.

Polymer Intermixed with Bioactive Agent or Additional Bioactive Agent

In addition to being linked to one or more bioactive agents, either directly or through a linker, a polymer used for coating a medical device or making a sheath for a stent structure as described herein can be physically intermixed with one or more bioactive agents or additional bioactive agents to provide a formulation.

As used herein, “intermixed” refers to a polymer of the present invention physically mixed with a bioactive agent or a polymer as described herein that is physically in contact with a bioactive agent.

As used herein, a “formulation” refers to a polymer as described herein that is intermixed with one or more bioactive agents or additional bioactive agents. The formulation includes such a polymer having one or more bioactive agents present on the surface of the polymer, partially embedded in the polymer, or completely embedded in the polymer. Additionally, the formulation includes a polymer as described herein and a bioactive agent forming a homogeneous composition (i.e., a homogeneous formulation).

By contrast, in the invention multilayered stents, in the outer layer non-covalently bound bioactive agents and/or additional bioactive agents can be intermingled with or “loaded into” any biocompatible biodegradable polymer as is known in the art since the outer layer in this embodiment of the invention does not come into contact with blood. However, the inner layer has only bioactive agents covalently attached to a hydrophilic, blood-compatible polymer as described herein.

Any suitable amount of polymer and bioactive agent can be employed to provide the formulation. The polymer can be present in about 0.1 wt. % to about 99.9 wt. % of the formulation. Typically, the polymer can be present above about 25 wt. % of the formulation; above about 50 wt. % of the formulation; above about 75 wt. % % of the formulation; or above about 90 wt. % of the formulation. Likewise, the bioactive agent can be present in about 0.1 wt. % to about 99.9 wt. % of the formulation. Typically, the bioactive agent can be present above about 5 wt. % of the formulation; above about 10 wt. % of the formulation; above about 15 wt. % of the formulation; or above about 20 wt. % of the formulation.

In yet another embodiment of the invention the polymer/bioactive agent, polymer/linker/bioactive agent, formulation, or combination thereof as described herein, can be applied, as a polymeric film onto the surface of a medical device (e.g., stent structure). The surface of the medical device can be coated with the polymeric film. The polymeric film can have any suitable thickness on the medical device. For example, the thickness of the polymeric film on the medical device can be about 1 to about 50 microns thick or about 5 to about 20 microns thick. In the invention stents and multilayered stents, each of the layers can be from 0.1 micron to 50 microns thick, for example from 0.5 micron to 5 microns in thickness.

The polymeric film can effectively serve as a bioactive agent eluting polymeric coating on a medical device, such as a stent structure. This bioactive agent eluting polymeric coating can be created on the medical device by any suitable coating process, e.g., dip coating, vacuum depositing, or spray coating the polymeric film, on the medical device. Additionally, the bioactive agent eluting polymer coating system can be applied onto the surface of a stent, a vascular delivery catheter, a delivery balloon, a separate stent cover sheet configuration, or a stent bioactive agent delivery sheath, as described herein to create a type of local bioactive agent delivery system.

The bioactive agent eluting polymer coated stents and other medical devices can be used in conjunction with, e.g., hydrogel-based bioactive agent delivery systems. For example, in one embodiment, the above-described polymer coated stents and medical devices, can be coated with an additional formulation layer applied over the polymer coated stent surface as a sandwich type of configuration to deliver to the blood vessels bioactive agents that promote natural re-endothelialization processes and prevent or reduce in-stent restenosis. Such an additional layer of hydrogel-based drug release formulation can comprise various bioactive agents mixed with hydrogels (see, U.S. Pat. No. 5,610,241, which is incorporated by reference herein in its entirety) to provide an elution rate different than that of the polymer/active agent coating on the stent structure or medical device surface.

Any suitable size of polymer and bioactive agent can be employed to provide such a formulation. For example, the polymer can have a size of less than about 1×10⁻⁴ meters, less than about 1×10⁻⁵ meters, less than about 1×10⁻⁶ meters, less than about 1×10⁻⁷ meters, less than about 1×10⁻⁸ meters, or less than about 1×10⁻⁹ meters.

The formulation can degrade to provide a suitable and effective amount of bioactive agent. Any suitable and effective amount of bioactive agent can be released and will typically depend, e.g., on the specific formulation chosen. Typically, up to about 100% of the bioactive agent can be released from the formulation. Specifically, up to about 90%, up to 75%, up to 50%, or up to 25% of the bioactive agent can be released from the formulation. Factors that typically affect the amount of the bioactive agent that is released from the formulation include, e.g., the nature and amount of polymer, the nature and amount of bioactive agent, and the nature and amount of additional substances present in the formulation.

The formulation can degrade over a period of time to provide the suitable and effective amount of bioactive agent. Any suitable and effective period of time can be chosen. Typically, the suitable and effective amount of bioactive agent can be released in about twenty-four hours, in about seven days, in about thirty days, in about ninety days, or in about one hundred and twenty days. Factors that typically affect the length of time in which the bioactive agent is released from the formulation include, e.g., the nature and amount of polymer, the nature and amount of bioactive agent, and the nature and amount of additional substances present in the formulation.

The present invention also provides for an invention stent coated with a formulation that includes a polymer as described herein physically intermixed with one or more bioactive agents. The polymer that is present in the formulation can also be linked, either directly or through a linker, to one or more (e.g., 1, 2, 3, or 4) bioactive agents. As such, the polymer can be intermixed with one or more (e.g., 1, 2, 3, or 4) bioactive agents and can be linked, either directly or through a linker, to one or more (e.g., 1, 2, 3, or 4) bioactive agents.

A polymer used in making an invention stent can include one or more bioactive agents. In one embodiment, the polymer is physically intermixed with one or more bioactive agents. In another embodiment, the polymer is linked to one or more bioactive agents, either directly or through a linker. In another embodiment, the polymer is linked to one or more bioactive agents, either directly or through a linker, and the resulting polymer can also be physically intermixed with one or more bioactive agents.

A polymer used in making an invention stent, whether or not present in a formulation as described herein, whether or not linked to a bioactive agent as described herein, and whether or not intermixed with a bioactive agent as described herein, can also be used in medical therapy or medical diagnosis. For example, the polymer can be used in the manufacture of a medical device. Suitable medical devices include, e.g., artificial joints, artificial bones, cardiovascular medical devices, stents, shunts, medical devices useful in angioplastic therapy, artificial heart valves, artificial by-passes, sutures, artificial arteries, vascular delivery catheters, drug delivery balloons, separate tubular stent cover sheet configurations (referred to herein as “sheaths”), and stent bioactive agent delivery sleeve types for local bioactive agent delivery systems.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

The invention will be further understood with reference to the following examples, which are purely exemplary, and should not be taken as limiting the true scope of the present invention as described in the claims.

### EXAMPLES

### Example 1

Amide Bond Formation—This example illustrates the coupling of a carboxyl group of a polymer with an amino functional group of the bioactive agent, or equally, the coupling of a carboxyl group of the bioactive agent with an amino functional group of a polymer.

Coupling Through Pre-Formed Active Esters; Carbodiimide Mediated Couplings—Conjugation of 4-Amino-Tempo to Polymer. The free carboxylic acid form of the PEA polymer is converted first to its active succinimidyl ester (PEA-OSu) or benzotriazolyl ester (PEA-OBt). This conversion can be achieved by reacting dried PEA-H polymer with N-Hydroxysuccinimide (NHS) or 1-Hydroxybenzotriazole (HOBt) and a suitable dehydrating agent, such as dicyclohexylcarbodiimide (DCC), in anhydrous CH₂Cl₂ at room temperature for 16 hrs. After filtering away the precipitated dicyclohexylurea (DCU), the PEA-OSu product may be isolated by precipitation, or used without further purification, in which case the PEA-OSu solution is transferred to a round bottom flask, diluted to the desired concentration, and cooled to 0° C. Next, a solution of the free amine-containing bioactive agent—the nucleophile, specifically, 4-Amino-Tempo—in CH₂Cl₂ is added in a single shot at 0° C. (Equally, the nucleophile may be revealed in situ by treating the ammonium salt of the bioactive agent with a hindered base, preferably a tertiary amine, such as like triethylamine or, diisopropylethylamine, in a suitable aprotic solvent, such as dichloromethane (DCM)). The reaction is monitored by tracking consumption of the free amine by TLC, as indicated by ninhydrin staining. Work-up for the polymer involves customary precipitation of the reaction solution into a mixture of non-solvent, such as hexane/ethyl acetate. Solvent is then decanted, polymer residue is resuspended in a suitable solvent, filtered, concentrated by roto-evaporation, cast onto a clean teflon tray, and dried under vacuum to furnish the PEA-bioactive agent conjugate, specifically, PEA-4-Amino-Tempo.

Aminium/Uronium Salt and Phosphonium Salt Mediated Couplings. Two effective catalysts for this type of coupling include: HBTU, O-(benzotriazol-1-yl)-1,1,3,3-teramethyluronium hexafluorophosphate, and BOP, 1-benzotriazolyoxytris(dimethyl-amino)phosphonium hexafluorophosphate (Castro's Reagent). These reagents are employed in the presence of equimolar amounts of the carboxyl group of the polymer and the amino functional group of the bioactive agent (neutral or as the ammonium salt), with a tertiary amine such as diisopropylethylamine, N-methylmorpholine, or dimethyl-substituted pyridines (DMAP), in solvents such as DMF, THF, or acetonitrile.

### Example 2

Ester Bond Formation—This example illustrates coupling of a carboxyl group of a polymer with a hydroxyl functional group of the bioactive agent, or equally, coupling of a carboxyl group of the bioactive agent with a hydroxyl functional group of a polymer.

Carbodiimide Mediated Esterification. For the conjugation, a sample of the carboxyl-group-containing polymer was dissolved in DCM. To this slightly viscous solution was added a solution of the hydroxyl-containing-drug/biologic and DMAP in DCM. The flask was then placed in an ice bath and cooled to 0° C. Next, a solution of 1,3-diisopropylcarbodiimide (DIPC) in DCM was added, the ice bath removed, and the reaction warmed to room temperature. The conjugation reaction was stirred at room temperature for 16 hours during which time TLC was periodically performed to monitor consumption of the hydroxyl functional group of the bioactive agent. After the allotted time, the reaction mixture was precipitated, and the Polymer-bioactive agent conjugate isolated as described above in Example 1.

### Example 3

This Example illustrates the effect of different concentrations of bioactive agents on adhesion and proliferation of epithelial cells (EC) and smooth muscle cells (SMC) on gelatin coated surfaces.

Human Coronary artery endothelial cells (EC) plated on gelatin coated culture plates were co-cultured with EC special media containing one of the bioactive agents shown in Table 1 below in the various concentrations shown.

TABLE 1

| | Bioagents | 100 μM | 10 μM | 1 μM | 100 nm |
|---|---|---|---|---|---|
| A | Bradykinin[Hyp 3] | 372 | 37.23 | 3.72 | 0.372 |
| B | Bradykinin | 322.8 | 32.28 | 3.228 | 0.3228 |
| C | Adenosine | 80.16 | 8.016 | 0.816 | 0.0816 |
| D | Sphingosine 1- | 113.85 | 11.385 | 1.1385 | 0.11385 |
| | Phosphate (S1P) | | | | |
| E | Lysophosphatidic | 137.55 | 13.755 | 1.375 | 0.1376 |
| | Acid (LPA) | | | | |
| F | Control | No | | | |
| | | additives | | | |

Cells cultured under similar conditions without adding bioagents are considered as ‘Control.’

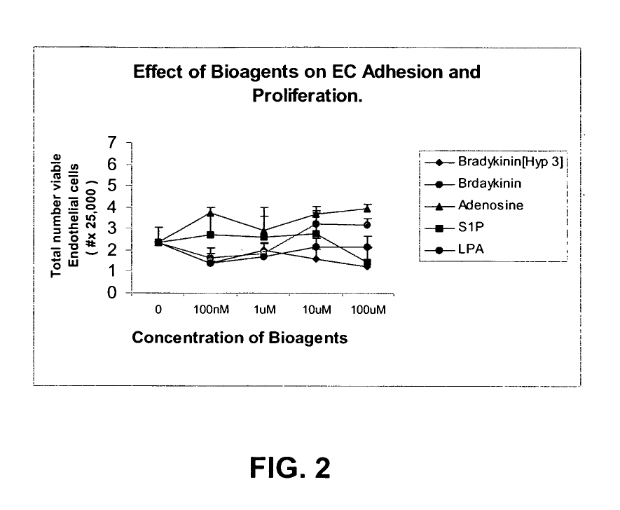

Twenty four hours later the cells were observed microscopically, stained with trypan blue and counted. The results of the microscopic observation of cell morphology and confluency of culturing the EC in the presence of the Bioagents tested are summarized in Table 2 below. The effect of the various bioagents on EC adhesion and proliferation is shown graphically in FIG. 2.

TABLE 2
Microscopic observation for the EC morphology and
Confluency in the presence of Bioagents

| Bioagents | 100 nm | 1 μM | 10 μM | 100 μM |
|---|---|---|---|---|
| Bradykinin | Normal Cell | Normal Cell | Normal Cell | Normal Cell |
| [Hyp 3] | Morphology and | Morphology and | Morphology and | Morphology and |
| | proliferation. | proliferation. | proliferation. | proliferation. |
| | Less confluent than | Less confluent | Less confluent | Less confluent than |
| | control | than control | than control | control |
| Bradykinin | Normal Cell | Normal Cell | Normal Cell | Normal Cell |
| | Morphology and | Morphology and | Morphology and | Morphology and |
| | proliferation. | proliferation. | proliferation. | proliferation. |
| | More confluent than | More confluent | Less confluent | Less confluent than |
| | control | than control | than control | control |
| Adenosine | Normal Cell | Normal Cell | Normal Cell | Normal Cell |
| | Morphology and | Morphology and | Morphology and | Morphology and |
| | proliferation. | proliferation. | proliferation. | proliferation. |
| | More confluent than | More confluent | More confluent | More confluent than |
| | control | than control | than control | control |
| S1P | ˜70% of cells | ˜50% of cells | 25% of cells | 95% of cells exhibited |
| | adhered with normal | adhered with | adhered with | distorted morphology. |
| | morphology and | normal | normal | No proliferating cells |
| | proliferation | morphology and | morphology and | Aggregates of dead |
| | | proliferation | proliferation. | cells were floating |
| | | | Lot of dead cells | |
| | | | were floating | |
| LPA | 70% cells Adhered | 50% cells | 30% cells | 10% cells Adhered and |
| | and exhibited | Adhered and | Adhered and | exhibited normal |
| | normal morphology. | exhibited normal | exhibited normal | morphology. |
| | Dead cells were | morphology. | morphology. | Big aggregates of dead |
| | floating | Dead cells were | Aggregates of | cells were floating |
| | | floating | dead cells were | |
| | | | floating | |
| Control | Normal | Normal | Normal | Normal Morphology, |
| | Morphology, and >85% | Morphology, and >85% | Morphology, and >85% | and >85% confluent |
| | confluent | confluent | confluent | |

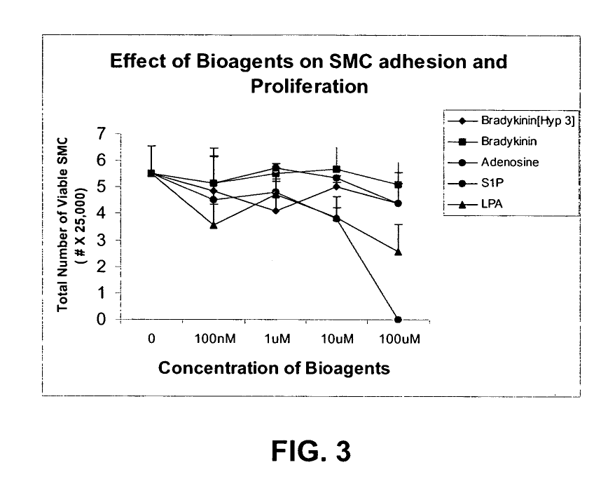

Effect of different concentrations of the bioagents listed above in Table 1 was also tested using human aortic smooth muscle cells (SMC) under similar conditions as described for EC. The results of the bioagents on adhesion and proliferation of SMC plated on gelatin coated culture plates are summarized in Table 3 below and shown graphically in FIG. 3.

TABLE 3
Microscopic observation for the SMC morphology and
Confluency in the presence of Bioagents

| Bioagents | 100 nm | 1 μM | 10 μM | 100? m |
|---|---|---|---|---|
| Bradykinin [Hyp 3] | Normal Cell | Normal Cell | Normal Cell | Normal Cell |
| | Morphology | Morphology | Morphology | Morphology |
| | and | and | and | and |
| | proliferation. | proliferation. | proliferation. | proliferation. |
| Bradykinin | Normal Cell | Normal Cell | Normal Cell | Distorted Cell |
| | Morphology | Morphology | Morphology | Morphology |
| | and | and | and | |
| | proliferation. | proliferation. | proliferation. | |
| | >70% confluent | 70% confluent | 50% confluent | |
| Adenosine | Normal Cell | Distorted Cell | 50% distorted | >50% distorted |
| | Morphology | Morphology | Cell | Cell |
| | and | | Morphology | Morphology |
| | proliferation. | | | |
| S1P | Normal | ˜50% cell | 70% cells | 100% cells died |
| | morphology. | adhered with | survived with | |
| | | normal | distorted | |
| | | morphology | morphology | |
| LPA | 70% cells | 50% cells | <50% cells | <10% cells |
| | Adhered and | Adhered and | Adhered and | Adhered and |
| | exhibited | exhibited | exhibited | exhibited |
| | normal | normal | normal | normal |
| | morphology. | morphology. | morphology. | morphology. |
| | | Lot of | | Big aggregates |
| | | dead cells were | | of dead cells |
| | | floating | | were floating |
| Control | Normal | Normal | Normal | Normal |
| | Morphology, | Morphology, | Morphology, | Morphology, |
| | and >85% | and >85% | and >85% | and >85% |
| | confluent | confluent | confluent | confluent |

### Example 4

This Example reports a pre-clinical animal model evaluation of the Blue Medical coronary stent stainless steel stent structure (Blue Medical Devices, BV, Helmund, the Netherlands) coated with TEMPO polymer, in three stages: 1) Evaluation of post-implantation injury and inflammatory response, 2) Evaluation of in-stent neointimal hyperplasia, and 3) Comparison of TEMPO coated stents with the uncoated stents.

Stent Implantation

Domestic crossbred pigs of both sexes weighing 20-25 kg were used for the study. The pigs were fed with a standard natural grain diet without lipid or cholesterol supplementation throughout the study. All animals were treated and cared for in accordance with the Belgium National Institute of Health Guide for the care and use of laboratory animals.

Acute Study—In the acute study 2 uncoated stents and 2 each of 5 types of coated stents with differently dosed coatings (0% TEMPO Gamma, 50% TEMPO Gamma, 0% TEMPO ETO, 50% TEMPO ETO, 100% TEMPO+Top Layer ETO) were randomly implanted in the coronary arteries of 6 pigs. Pigs were sacrificed after 5 days to evaluate acute inflammatory response and thrombus formation caused by implantation of the stents. TEMPO=stent coated with 4-amino Tempo in polymer; (Gamma=stent sterilized with gamma radiation; and ETO=stent sterilized with ethylene oxide.

Chronic Study—In this study 8 uncoated stents and 8 TEMPO coated stents, 4 with 50% TEMPO and 4 wityh 100% TEMPO, were randomly implanted in the coronary arteries of selected pigs. The pigs were sacrificed after 6 weeks to evaluate peri-strut inflammation and neointimal hyperplasia. Surgical procedure and stent implantation in the coronary arteries were performed according to the methods described by De Scheerder et al. (Atherosclerosis. (1995) 114:105-114 and Coron Artery Dis. (1996) 7:161-166.

Prior to stent implantation, a balloon catheter was used as a reference to expand the stents to obtain an over-sizing of the artery of 10% to 20%, thereby causing damage to endothelium.

Quantitative Coronary Angiography—Angiographic analysis of stented vessel segments was performed before stenting, immediately after, and at follow-up using the polytron 1000®-system as described previously by De Scheerder et al. The diameter of the vessel segments was measured before and immediately after stent implantation, and at follow-up 6 weeks after implantation. The degree of over-sizing was expressed as measured maximum balloon size minus selected artery diameter divided by selected artery diameter.

Histopathology and Morphometry—Coronary segments were carefully dissected, leaving a 1 cm minimum vessel length attached both proximal and distal to the stent. The segments were fixed in a 10% formalin solution. Each segment was cut into proximal, middle and distal stent segments for histomorphometric analysis. Tissue specimens were embedded in a cold-polymerizing resin (Technovit 7100, Heraus Kulzer GmbH, Wehrheim, Germany). Sections 5 microns thick were cut with a rotary heavy duty microtome (HM 360, Microm, Walldorf, Germany) equipped with a hard metal knife and stained with hematoxylin-eosin, elastic stain and with phosphotungstic acid hematoxylin stain. Examination was performed using a light microscope by an experienced pathologist, who was blinded to the type of stent inspected. Injury of the arterial wall due to stent deployment (and eventually inflammation induced by the polymer) was evaluated for each stent filament and graded as described by Schwartz et al. (J Am Coll Cardiol 1992;19(2):267-74).

Grade 0=internal elastic membrane intact, media compressed but not lacerated;

Grade 1=internal elastic membrane lacerated; Grade 2=media visibly lacerated;

external elastic membrane compressed but intact; Grade 3=large laceration of the media extending through the external elastic membrane or stent filament residing in the adventitia.

Inflammatory reaction at each stent filament was carefully examined, searching for inflammatory cells, and scored as follows:

1=sparsely located histolymphocytes surrounding the stent filament; 2=more densely located histolymphocytes covering the stent filament, but no lymphogranuloma and/or giant cells formation found; 3=diffusely located histolymphocytes, lymphogranuloma and/or giant cells, also invading the media.

The mean score for each stent was calculated by summing the score for each filament and dividing by the number of filaments present.

Morphometric analysis of the coronary segments harvested was performed using a computerized morphometry program (Leitz CBA 8000). Measurements of lumen area, lumen area inside the internal elastic lamina, and lumen inside the external elastic lamina were performed. In addition, the areas of stenosis and neointimal hyperplasia were calculated.

Statistics—For comparison among different groups, non-paired t-test was used. Data are presented as mean value ±SD. A p value ≦0.05 was considered as statistically significant.

Results

Quantitative Coronary Angiography—As the number of stents used for the acute study was limited, the acute study stents were grouped with those from the chronic study to evaluate the degree of over-sizing that occurred. Angiographic measurements showed that the selected arterial segments and recoil ratio of TEMPO coated groups were similar to those for the bare control group (Table 4 below). The balloon size of the 0% TEMPO Gamma, 50% TEMPO ETO, and 100% TEMPO+Top Layer ETO groups was significantly lower than the balloon size for the bare stent groups. However, no significant difference in over-sizing was observed in different groups as compared to the bare stent groups.

TABLE 4
Quantitative coronary angiography

| | | Pre- | | Post- | Recoil | |
|---|---|---|---|---|---|---|
| | | stenting | Balloon size | stenting | ratio** | Over-sizing |
| | N | (mm) | (mm) | (mm) | (%) | (%) |
| Bare stent | 9 | 2.63 ± 30 | 3.17 ± 0.26 | 3.09 ± 0.28 | 2.51 ± 2.34 | 21.26 ± 9.00 |
| 0% TEMPO Gamma | 10 | 2.59 ± 0.13 | 2.96 ± 0.06* | 2.88 ± 0.09 | 2.91 ± 2.05 | 14.60 ± 4.73 |
| 50% TEMPO Gamma | 9 | 2.66 ± 0.23 | 3.02 ± 0.11 | 2.92 ± 0.14 | 3.37 ± 1.83 | 14.31 ± 6.91 |
| 0% TEMPO ETO | 9 | 2.47 ± 0.16 | 2.97 ± 0.07 | 2.86 ± 0.06* | 3.76 ± 1.89 | 20.43 ± 2.65 |
| 50% TEMPO ETO | 8 | 2.52 ± 0.14 | 2.95 ± 0.12* | 2.84 ± 0.14* | 3.87 ± 3.56 | 17.22 ± 3.72 |
| 100% TEMPO + Top | 9 | 2.42 ± 0.12 | 2.93 ± 0.10* | 2.84 ± 0.10* | 3.02 ± 2.32 | 21.02 ± 5.72 |
| Layer ETO | | | | | | |

*Comparing to bare stent group, P < 0.05
**Recoil ratio = (1 − minimal lumen diameter immediately after implantation/maximal balloon diameter) × 100 (%)

Histopathology

At 5 days follow-up, residual polymer material was detected around the stent filaments. The inflammatory response of all TEMPO coated stents and bare stents was low: (0% TEMPO Gamma, 1.00+0.00; 50% TEMPO Gamma, 1.00+0.00; 0% TEMPO ETO, 1.06+0.10; 50% TEMPO ETO, 1.00+0.00; and 100% TEMPO+Top Layer ETO, 1.00+0.00 compared with bare stents (1.03±0.07). A few inflammatory cells were seen adjacent to the stent filaments. Stent struts with moderate inflammatory reaction were rare. A thin thrombotic meshwork covering the stent filaments was observed. Internal elastic lamina membrane was beneath the stent filaments and the media was moderately compressed. Arterial injury caused by stent deployment was low and identical for the groups (0% TEMPO Gamma, 0.24±0.10; 50% TEMPO Gamma, 0.32±0.18; 0% TEMPO ETO, 0.28+0.01; 50% TEMPO ETO, 0.25±0.01; 100% TEMPO+Top Layer ETO, 0.13±0.08; and bare stents, 0.19+0.13).

At 6 weeks follow-up, disruption of internal elastic lamina was often seen in the bare stent group. In some sections, a few stent struts lacerated external elastic lamina and even penetrated into the adventitia. In the TEMPO coated stent groups, stent struts compressed the arterial medial layer. Some internal elastic lamina was lacerated. Only a few sections showed a disruption of arterial media and/or external elastic lamina. Compared to bare stent group, the mean injury scores of the TEMPO coated stent groups were decreased (Table 2). Furthermore, the TEMPO coated stent groups showed only a mild inflammatory response. Spare inflammatory cells were observed around the stent struts. Several stent struts showed a moderate inflammatory response. No inflammatory cells were found infiltrated into media. The mean inflammatory scores of 0% TEMPO GAMMA, 50% TEMPO GAMMA and 50% TEMPO ETO groups were significantly lower than for the bare stent group.

Morphometry

At 6 weeks follow-up (as shown in Table 5 below), the lumen area of 100% TEMPO+Top Layer ETO was the smallest among the groups. Compared to the lumen area of the bare stent group, however, no significant difference was observed (4.29±2.28 vs 3.60+0.99, P>0.05). The neointimal hyperplasia and area stenosis of all TEMPO groups were lower than those for the bare stent group, but only the 0% TEMPO Gamma and the 50% TEMPO Gamma groups showed a significant decrease in neointimal hyperplasia and area stenosis. The neointimal hyperplasia of the 50% TEMPO Gamma group was the lowest.

TABLE 5
Histomorphometric analysis of stented vessel segments at 6 weeks follow-up

| | | Lumen | Neointimal | Area | | |
|---|---|---|---|---|---|---|
| | | Area | Hyperplasia | Stenosis | Inflammation | |
| | N | (mm²) | (mm²) | (%) | Score | Injury Score |
| Bare stent | 24 | 4.29 ± 2.28 | 1.78 ± 0.79 | 35 ± 23 | 1.09 ± 0.14 | 0.62 ± 0.46 |
| 0% TEMPO | 24 | 4.45 ± 0.90 | 1.26 ± 0.41* | 23 ± 9* | 1.02 ± 0.05* | 0.34 ± 0.18** |
| Gamma | | | | | | |
| 50% TEMPO | 24 | 4.31 ± 0.70 | 1.10 ± 0.18** | 21 ± 4* | 1.02 ± 0.05* | 0.39 ± 0.27* |
| Gamma | | | | | | |
| 0% TEMPO ETO | 24 | 4.15 ± 0.82 | 1.42 ± 0.61 | 26 ± 11 | 1.03 ± 0.07 | 0.31 ± 0.24** |
| 50% TEMPO ETO | 24 | 4.03 ± 0.78 | 1.36 ± 0.51 | 26 ± 10 | 1.01 ± 0.04* | 0.30 ± 0.18** |
| 100% TEMPO + Top | 24 | 3.60 ± 0.99 | 1.47 ± 0.68 | 30 ± 14 | 1.04 ± 0.07 | 0.46 ± 0.26 |
| Layer ETO | | | | | | |
| Bare stent | 24 | 4.29 ± 2.28 | 1.78 ± 0.79 | 35 ± 23 | 1.09 ± 0.14 | 0.62 ± 0.46 |
| 0% TEMPO | 48 | 4.24 ± 0.91 | 1.41 ± 0.61* | 25 ± 11* | 1.03 ± 0.06* | 0.33 ± 0.21** |
| 50% TEMPO | 48 | 4.13 ± 0.69 | 1.27 ± 0.42** | 24 ± 8** | 1.02 ± 0.05** | 0.34 ± 0.23** |
| 100% TEMPO + Top | 24 | 3.60 ± 0.99 | 1.47 ± 0.68 | 30 ± 14 | 1.04 ± 0.07 | 0.46 ± 0.26 |
| Layer ETO | | | | | | |

Comparing to bare stent group, * = P < 0.05, ** = P < 0.01

Conclusion

The TEMPO coated and bare stents elicited a similar tissue response at 5 days follow-up. No additional inflammatory response or increased thrombus formation was observed for the TEMPO coated stents at that time point. At 6 weeks follow-up, the neointimal formation induced by the TEMPO coated stent groups was lower than for the bare stent group. Both area stenosis and neointimal hyperplasia of 0% TEMPO Gamma and 50% TEMPO Gamma-coated stents were significantly lower than for the bare stent group. In addition, a significantly decreased peri-strut inflammation for the 0% TEMPO GAMMA, 50% TEMPO GAMMA and 50% TEMPO ETO-coated stents was observed as compared to the bare stent group. In conclusion, The TEMPO coating did not induce an increased tissue response. TEMPO coated stents sterilized with Gamma radiation showed a beneficial effect on neointimal formation at 6 weeks follow-up, especially in the 50% TEMPO group. Increased TEMPO loaded concentrations or/and addition of a top layer of de-protected polyester amide polymer—PEA(H)— did not show a consistent inhibitory effect on neointimal hyperplasia and area stenosis.

### Example 5

Noblesse Clinical Trial

Study Design

The Noblesse (Nitric Oxide through Biodegradable Layer Elective Study for Safety and Efficacy) Clinical Trial was conducted in human patients to determine the effects of implantation in a human of a functionalized polymer coating on a coronary stent without the presence of a drug. The stent used was the Genic stainless steel stent structure (Blue Medical Devices, BV, Helmund, the Netherlands) coated with PEA-Tempo, (Poly(Ester)Amide—4 amine Tempo) functionalized polymer (MediVas LLC, San Diego, Calif.).

The clinical trial was a multi-center, prospective, non-randomized study of forty five patients that included angiographic follow-up at four months and angiographic and IVUS follow-up at twelve months. The study took place in three locations: Cordoba, Argentina, Curitiba, Brazil and Eindhoven, the Netherlands.

All patients were provided with a written informed consent prior to enrollment in the study. Patients were required to have stable or unstable angina pectoris or a positive exercise test, be at least eighteen years old, have a single, de-novo target lesion in native coronary artery, have the reference vessel be visually estimated to be greater than 2.75 mm and less than 3.50 mm in diameter, have target lesion stenosis greater than 50% and less than 100%, and have a target lesion less than 15 mm in length.

The primary endpoint of the study was the late loss of the luminal area at four months and twelve months after stent placement. Secondary endpoints were 30 day, 60 day, 120 day, and 12 month MACE (major arterial coronary event), death, recurrent myocardial infarction, or target lesion revascularization (requiring re-stenting).

Prior to the implantation procedure, each patient received at least 100 mg aspirin before stenting and oral clopidogrel of 300 mg before PTCA. Each patient received intracoronary nitroglycerin of 50-200 μg prior to baseline angiography, during post-stent deployment and after final post dilatation angiography. Each patient also received sufficient heparin to maintain ACT of 250-300 seconds. For 28 days after the procedure each patient received 75 mg/d of Clopidogrel.

Patient Demographics

Of the forty-five patients, thirty-one (69%) were male. The patients ranged in age from 38 to 83 years, with a mean age of 62 years. Twenty-two patients were enrolled in Brazil, eighteen in Argentina, and five in the Netherlands.

Lession Characteristics:

| | |
|---|---|
| The vessel in the heart treated in patients | |
| Right coronary artery | 40.0% |
| Left anterior descending artery | 7.5% |
| Left circumflex artery | 22.5% |
| AHA/ACC class^{a} | |
| A: | 14.3% |
| B1: | 61.9% |
| B2: | 23.8% |
| TIMI 3 (a blood flow measure)^{b} | 100% |
| Angulation >45%^{c} | 19.1% |
| Moderate vessel tortuousity^{d} | 23.8% |
| Avg. Ref Vessel Diameter:^{e} | 2.98 ± 0.32 mm |
| Avg. Minimum luminal diameter prior to stenting:^{f} | 1.05 ± 0.34 mm |
| Avg. Minimum luminal diameter 4 mos. after | 2.74 ± 0.26 mm |
| stenting:^{g} | |
| Avg. Diameter of Stenosis prior to stenting:^{h} | 64.69 ± 11.59% |
| Avg. Diameter Stenosis 4 mos. after stenting:ⁱ | 8.70 ± 4.52% |
| Avg. Acute Gain^{j} | 1.69 ± 0.42 mm |
| All patients were discharged 24 hours after | |
| the procedure with no complications. | |
| Cardiac death | 0 |
| Q-wave MI (as read by electrocardiogram)^{k} | 0 |
| Non Q-wave MI | 0 |
| CABG required^{l} | 0 |
| TLR* | 0 |
| At the twelve month follow-up patient results were | |
| as follows: | |
| Cardiac death | 0 |
| Q-wave MI (as read by electrocardiogram) | 0 |
| Non Q-wave MI | 0 |
| Coronary artery bypass surgery required | 0 |
| TLR^{m} | 1 |
| Average minimum luminal diameter at 12 months | 2.87 ± 0.31 mm |
| post stenting:: | |

^{a}The AHA/ACC class refers to the American Heart Association/American College of Cardiology rating system for severity of blockage. The severity increases from mild (A1) through moderate (B1) to severe (B2). Total occlusion is C.
^{b}TIMI 3 refers to thrombolysis in myocardial infarction. These are a rating of the blood's ability to flow, going from 1 to 3, with 3 being the most flow (or least likely to have thrombosis). TIMI 4 is total occlusion.
^{c}Angulation >45% means the percentage of target arteries that have a bend of 45% or more within the target lesion.
^{d}Moderate vessel tortuousity (slide 5) is an objective evaluation by the interventionalist as to the degree of “twistiness” of the artery.
^{e}Ref Vessel Diameter is the size of the native artery immediately proximal to the target lesion.
^{f}MLD Pre (means “minimum luminal diameter” and describes the smallest cross section of the artery at the lesion site prior to stent placement.
^{g}MDL Post means “minimum luminal diameter” and describes the smallest cross section of the artery at the lesion site after stent placement.
^{h}Diameter Stenosis Pre is calculated by subtracting MLD Pre from Ref Vessel Diameter and dividing by Ref Vessel Diameter.
ⁱDiameter Stenosis Post is calculated by subtracting MLD Post from Ref Vessel Diameter and dividing by Ref Vessel Diameter.
^{j}Acute gain is Diameter Stenosis Pre-subtracted from Diameter Stenosis Post.
^{k}Q-wave MI and Non Q-wave MI are two forms of myocardial infractions (heart attacks) as indicated by electrocardiogram.
^{l}CABG is coronary artery bypass graph and refers to bypass surgery.
^{m}TLR is total lesion revascularization and refers to the placement of a second stent to correct the failure of the first stent.

Conclusions

The PEA-4 Amine Tempo polymer was shown to be a safe form of bioabsorbable polymer and the polymer alone, without added drug, demonstrated a unique capability to preserve and even enhance the beneficial effect of the invention stents in coronary arteries as measured by the increase in average minimum luminal diameter in treated heart arteries 12 months after stent emplacement.

Although the invention has been described with reference to the above examples, it will be understood that modifications and variations are encompassed within the spirit and scope of the invention. Accordingly, the invention is limited only by the following claims.

What is claimed is:

1. A bioactive implantable stent comprising a stent structure with a surface coating of a biodegradable, bioactive polymer, wherein the polymer comprises at least one bioactive agent covalently bound to the polymer, and wherein the at least one bioactive agent produces a therapeutic effect in situ.

2. The stent of claim 1, wherein the at least one bioactive agent is produced in situ as a result of biodegradation of the polymer.

3. The stent of claim 1, wherein the stent is sized for implanting in the vasculature and promotes endogenous wound healing processes at a site of implantation.

4. The stent of claim 1, wherein the at least one bioactive agent is selected to promote production of nitric oxide by endothelial cells at a locus of endothelial damage to a vessel and/or control proliferation of smooth muscle cells in the vessel at the locus of the damage.

5. The stent of claim 4, wherein the at least bioactive agent donates, transfers or releases nitric oxide, elevates endogenous levels of nitric oxide, stimulates endogenous synthesis of nitric oxide, or serves as a substrate for nitric oxide synthase.

6. The stent of claim 5, wherein the at least one bioactive agent is selected from arginine, lysine, aminoxyls, furoxans, nitrosothiols, nitrates, anthocyanins, sphingosine-1-phosphate, phospholipid lysophosphatidic acid.

7. The stent of claim 5, wherein the at least one bioactive agent is arginine.

8. The stent of claim 5, wherein the at least one bioactive agent is sphingosine-1-phosphate.

9. The stent of claim 5, wherein the at least one bioactive agent is phospholipid lysophosphatidic acid.

10. The stent of claim 5, wherein the at least one bioactive agent is an aminoxyl.

11. The stent of claim 10, wherein the aminoxyl is 4-amino-2,2,6,6-tetramethylpiperidinyloxy, free radical (4-Amino-TEMPO).

12. The stent of claim 1, wherein the polymer is polyester, poly(amino acid), polyester amide, polyurethane, or copolymers thereof.

13. The stent of claim 12, wherein the polyester is polylactide, polylactone, poly(α-hydroxy-carboxylic acid), poly(glycolic acid), or poly(3-hydroxybutyrate), or copolymers thereof.

14. The stent of claim 13, wherein said polylactone is polycaprolactone.

15. The stent of claim 1, wherein the bioactive agent is attached to the biodegradable, bioactive polymer via a linker.

16. The stent of claim 15, wherein the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀)aryl, and W and Q are each independently —N(R)C(═O)—, —C(═O)N(R)—, —OC(═O)—, C(═O)O, —O—, —S—, —S(O), —S(O)₂—, —S—S—, —N(R)—, —C(═O)—, wherein each R is independently H or (C₁-C₆)alkyl.

17. The stent of claim 15, wherein the linker is a polypeptide comprising 2 up to about 25 amino acids.

18. The stent of claim 17, wherein said polypeptide is poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, or poly-L-lysine-L-tyrosine.

19. The stent of claim 15, wherein the linker separates the bioactive agent from the biodegradable, bioactive polymer by about 5 angstroms up to about 200 angstroms.

20. A bioactive vascular stent comprising a stent structure with a surface coating of a biodegradable, bioactive polymer, wherein at least one ligand for promoting re-endothelialization of endothelial cells is covalently bonded to the polymer.

21. The stent of claim 20, wherein the ligand is selected from peptides that promote endothelial cell growth.

22. The stent of claim 21, wherein the peptides that promote endothelial cell growth are selected from protein A and protein G.

23. The stent of claim 21, wherein the ligand is Protein A having an amino acid sequence as set forth in SEQ ID NO:1 or SEQ ID NO:2.

24. The stent of claim 21, wherein the ligand is Protein G having an amino acid sequence as set forth in SEQ ID NO:3 or SEQ ID NO:4.

25. The stent of claim 21, wherein the ligand is selected from bradykinins 1 and 2.

26. The stent of claim 20, wherein the polymer comprises polyester, poly(amino acid), polyester amide, polyurethane, or copolymers thereof.

27. The stent of claim 26, wherein the polyester is polylactide, polylactone, poly(α-hydroxy-carboxylic acid), poly(glycolic acid), or poly(3-hydroxybutyrate), or copolymers thereof.

28. The stent of claim 27, wherein the polylactone is polycaprolactone.

29. The stent of claim 20, wherein the bioactive agent is attached to the biodegradable, bioactive polymer via a linker.

30. The stent of claim 29, wherein the linker is a divalent radical of formula W-A-Q, wherein A is (C₁-C₂₄)alkyl, (C₂-C₂₄)alkenyl, (C₂-C₂₄)alkynyl, (C₃-C₈)cycloalkyl, or (C₆-C₁₀)aryl, and W and Q are each independently —N(R)C(═O)—, —C(═O)N(R)—, —OC(═O)—, —C(═O)O, —O—, —S—, —S(O), —S(O)₂—, —S—S—, —N(R)—, —C(═O)—, wherein each R is independently H or (C₁-C₆)alkyl.

31. The stent of claim 29, wherein the linker is a polypeptide comprising 2 up to about 25 amino acids.

32. The stent of claim 31, wherein said polypeptide is poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, or poly-L-lysine-L-tyrosine.

33. The stent of claim 29, wherein the linker separates the bioactive agent from the biodegradable, bioactive polymer by about 5 angstroms up to about 200 angstroms.

34. The stent of claim 1 or 17, wherein the stent is sized for intravascular insertion.

35. A tubular sheath comprising a biodegradable, bioactive polymer, wherein the polymer comprises at least one bioactive agent covalently bound to the polymer.

36. The sheath of claim 35, wherein the at least one bioactive agent is produced in situ as a result of biodegradation of the polymer.

37. The sheath of claim 35, wherein the biodegradable, bioactive polymer is elastomeric.

38. A bioactive implantable stent comprising a stent structure with a surface coating of a biodegradable, bioactive polymer, wherein the polymer produces a therapeutic effect in situ as a result of biodegradation of the polymer.

39. A biodegradable stent, wherein the stent comprises a cross-linked biodegradable polymer.

40. The biodegradable stent of claim 39, wherein the crosslinked biodegradable polymer is poly(caprolactone), poly(ester ether), poly(ester urethane), or a combination thereof.

41. A method for promoting natural healing of a damaged artery comprising implanting into the artery a stent according to claim 1 or 17 under conditions suitable for promoting natural healing of the artery.

42. The method of claim 41, wherein the natural healing comprises re-endothelialization of the artery wall.

43. A method of using a polymer as a medical device, a pharmaceutical, or as a carrier for covalent immobilization of a bioactive agent, wherein the polymer comprises at least one bioactive agent covalently bound to the polymer

44. The method of claim 43, wherein the at least one bioactive agent is produced in situ as a result of biodegradation of the polymer.

45. The method of claim 42, wherein the polymer coats an implantable medical device and the bioactive agent promotes natural wound healing processes in situ by contact with surrounding body area into which the medical device is implanted.

46. A bioactive implantable stent comprising:
a porous stent structure; and
a multilayered tubular coating encapsulating the stent structure, the multilayered coating comprising:
an outer drug-eluting biodegradable polymer layer, which sequesters an unbound drug; and
an inner layer of a biodegradable, bioactive polymer, wherein the polymer comprises at least one bioactive agent covalently bound to the polymer, and wherein the at least one bioactive agent produces a therapeutic effect in situ; and
an biodegradable barrier layer lying between and in contact with the outer layer and the inner layer and which barrier layer is impermeable to the drug.

47. The stent of claim 46, wherein the at least one bioactive agent comprises a ligand that promotes re-endothelialization of endothelial cells is covalently bonded to the inner polymer layer.

48. The stent of claim 47, wherein the ligand is selected from peptides that promote endothelial cell growth.

49. The stent of claim 48, wherein the peptides that promote endothelial cell growth are selected from protein A and protein G.

50. The stent of claim 48, wherein the ligand comprises Protein A having an amino acid sequence as set forth in SEQ ID NO:1 or SEQ ID NO:2.

51. The stent of claim 48, wherein the ligand comprises Protein G having an amino acid sequence as set forth in SEQ ID NO:3 or SEQ ID NO:4.

52. The stent of claim 47, wherein the ligand is selected from bradykinins 1 and 2.

53. The stent of claim 46, wherein the bioactive agent is attached to the biodegradable, bioactive polymer in the inner layer via a linker.

54. The stent of claim 53, wherein the linker is a polypeptide comprising 2 up to about 25 amino acids.

55. The stent of claim 54, wherein said polypeptide is poly-L-lysine, poly-L-glutamic acid, poly-L-aspartic acid, poly-L-histidine, poly-L-ornithine, poly-L-threonine, poly-L-tyrosine, poly-L-leucine, poly-L-lysine-L-phenylalanine, poly-L-arginine, or poly-L-lysine-L-tyrosine.

56. The stent of claim 46, further comprising an additional bioactive agent.

57. The stent of claim 56 wherein the additional bioactive agent is rapamycin, paclitaxel, everolimus, a statin, or an analog or derivative thereof.

58. The stent of claim 57, wherein the drug is hydrophobic and the barrier layer is less hydrophobic than the drug.

59. The stent of claim 57, wherein the drug is hydrophilic and the barrier layer is hydrophobic.

60. The stent of claim 58 or 59 wherein the polymer barrier layer comprises polyester, poly(amino acid), poly(ester amide), poly(esterurethane), polyurethane, polylactone, poly(ester ether), or copolymers thereof.

61. The stent of claim 46, wherein the stent is sized for intravascular insertion.

62. A method for promoting natural healing of an artery having an endothelium damaged by mechanical intervention comprising implanting into the artery immediately following the mechanical intervention a stent according to claim 1 or 46 under conditions suitable for promoting natural healing of the artery.

63. The method of claim 62, wherein the natural healing comprises re-endothelialization of the artery.

64. The method of claim 63, wherein the stent physically impairs activation of smooth muscle cells.

65. The method of claim 64, wherein the mechanical intervention is angioplasty.

66. The method of claim 62, wherein the mechanical intervention is balloon angioplasty.

67. The method of claim 62, wherein the method comprises implanting the stent to substantially cover a section of the interior artery wall damaged by the mechanical intervention.


SEQUENCE LISTING (8 sequences)

SEQ ID NO: 1 - protein, 61 aa, Artificial sequence [sequence omitted]

SEQ ID NO: 2 - protein, 55 aa, Artificial sequence
  Small bacterial proteinaceous motif
TYKLILNGKTLKGETTTEAVDAATAEKVFKQYANDNGVDGEWTYDDATKTFTVTE

SEQ ID NO: 3 - protein, 61 aa, Artificial sequence [sequence omitted]

SEQ ID NO: 4 - protein, 55 aa, Artificial sequence; identical to SEQ ID NO: 2
  Synthetic peptide

SEQ ID NO: 5 - protein, 10 aa, Artificial sequence
  Small proteinaceous motif
KRPPGFSPFR

SEQ ID NO: 6 - protein, 9 aa, Artificial sequence
  Small proteinaceous motif
KRPPGFSPF

SEQ ID NO: 7 - protein, 9 aa, Artificial sequence
  Small proteinaceous motif
RPPGFSPFR

SEQ ID NO: 8 - protein, 8 aa, Artificial sequence
  Small proteinaceous motif
RPPGFSPF